(19) United States
(12) Patent Application Publication
(10) Pub. No.: US 2022/0275061 A1
(43) Pub. Date: Sep. 1, 2022

(54) THERAPEUTIC PROTEIN FORMULATIONS COMPRISING ANTIBODIES AND USES THEREOF

(71) Applicant: CONTRAFECT CORPORATION, Yonkers, NY (US)

(72) Inventor: Abraham THARIATH, San Mateo, CA (US)

(21) Appl. No.: 17/625,916

(22) PCT Filed: Jul. 10, 2020
(86) PCT No.: PCT/US2020/041687

§ 371 Date: Jan. 10, 2022

Related U.S. Application Data
(60) Provisional application No. 62/873,749, filed on Jul. 12, 2019.

(51) Int. Cl. C07K 16/10; A61P 31/16

(52) U.S. Cl. CPC C07K 16/1018; A61P 31/16; A61K 2039/505

(57) ABSTRACT

The present disclosure is directed to an aqueous therapeutic protein formulation including: (i) one or more therapeutic proteins, wherein the one or more therapeutic proteins include one or more anti-influenza antibodies or antigen-binding fragments thereof in an amount ranging from 30 to 150 mg/mL; (ii) histidine buffer, (iii) NaCl, and (iv) an aqueous carrier, wherein a pH of the aqueous therapeutic formulation ranges from 5.5-8.0, and wherein the formulation is formulated for respiratory tract delivery and produces particles including the one or more therapeutic proteins upon aerosolization. Methods of generating an aerosol and treating influenza are also provided.

### CROSS-REFERENCE TO RELATED APPLICATIONS

This application claims the benefit of, and relies on the filing date of, U.S. provisional patent application No. 62/873,749, filed 12 Jul. 2019, the entire contents of which are incorporated herein by reference.

### SEQUENCE LISTING

The instant application contains a Sequence Listing which has been submitted electronically in ASCII format and is hereby incorporated by reference in its entirety. Said ASCII copy, created on 8 Jul. 2020, is named 0341_0006-00-304_SL.txt and is 110,998 bytes in size.

### FIELD

Formulations comprising therapeutic protein(s) suitable for aerosolization and treatment of influenza via administration of the respiratory tract are provided.

### BACKGROUND

Monoclonal antibodies (mAbs) and other antibody-based therapies have proven successful for the treatment of cancers, inflammatory and autoimmune diseases. More recently, antibody cocktails have been developed that are effective for treating influenza, including influenza B. While most antibodies are administered via the blood, less invasive routes of administration are currently being explored for the treatment of influenza as well as long-term chronic diseases. For respiratory diseases, the airways are a possible route for the local delivery of drugs and this route is routinely used in clinical practice for the delivery of small drug molecules, such as β2-adrenoreceptor agonists, muscarinic antagonists, and corticosteroids. The airways have recently been evaluated for the delivery of biopharmaceuticals, including antibodies. However, administration of proteins by inhalation is rare and only one protein drug, dornase alfa (PULMOZYME®), a recombinant human DNase used for the treatment of cystic fibrosis, is currently approved.

The respiratory tract delivery of antibodies is challenging in terms of the formulation of biological agents for inhalation. A prerequisite for successful inhalation therapy is the efficient distribution and reliable deposition of sufficient numbers of aerosol particles in the respiratory tract region of interest. This is dependent on aerosol technology, the performance of the device (e.g., aerosol output, particle size) and the physical characteristics of the drug formulation. Nebulizers are the most widely used inhalers for generating aerosols from protein solutions. However, while nebulizers are recognized as useful for generating aerosols from protein solutions, the effect of aerosolization and protein formulation on the molecular integrity of an active antibody may prohibit effective inhalation therapies. Like other therapeutic proteins, antibodies may undergo conformational changes, potentially decreasing their biological activity. Further, antibodies are susceptible to various stresses, such as high temperature, extreme pH, shear stress and freezing. Aerosol formation involves the dispersion/suspension of solid material or liquid droplets in a gaseous medium. This process is associated with physical stresses likely to induce changes in protein conformation. The development of inhaled antibody treatments is therefore a challenge for drug formulators.

### SUMMARY

The present disclosure is directed to a therapeutic protein formulation, typically comprising antibodies, which is particularly suitable for treating influenza. The formulation, which may be aerosolized and delivered into the respiratory tract, reduces the possibility of e.g., coughing and irritation of the lung mucosa. Moreover, the formulation is particularly useful for stabilizing therapeutic proteins, such as antibodies, thereby mitigating loss of activity during storage or aerosolization. Furthermore, the formulation may include relatively high concentrations of therapeutic protein, which reduces the need for large volumes of formulation and prolonged aerosolization times and, thus, typically promotes patient compliance. These and other unexpected benefits of the present formulation are described herein.

In one aspect, the present disclosure is directed to an aqueous therapeutic protein formulation comprising: (i) one or more therapeutic proteins, wherein the one or more therapeutic proteins comprise one or more anti-influenza antibodies or antigen-binding fragments thereof in an amount ranging from 30 to 150 mg/mL; (ii) histidine buffer, (iii) NaCl, and (iv) an aqueous carrier, wherein a pH of the aqueous therapeutic formulation ranges from 6.0-8.0, and wherein the formulation is formulated for respiratory tract delivery and produces particles comprising the one or more therapeutic proteins upon aerosolization. In certain embodiments, the aqueous therapeutic protein formulation further comprises a surfactant, such as a polysorbate.

Also provided herein is a method of generating an aerosol comprising the step of: nebulizing the aqueous therapeutic protein of any one of the preceding claims using a nebulizer to obtain an aerosol.

In another aspect, the present disclosure provides a method for the therapeutic and/or prophylactic treatment of influenza, which method comprises administering the aqueous therapeutic protein formulation to a subject in need thereof.

### BRIEF DESCRIPTION OF THE DRAWINGS

FIGS. 1A-1B depict histograms of particle size distribution for a formulation of the disclosure containing 20 mM histidine-chloride buffer and 115 mM NaCl as described in the Examples.

FIGS. 2A-2B depict histograms of particle size distribution for a formulation of the disclosure containing 20 mM histidine-chloride buffer, 115 mM NaCl and 0.02% polysorbate-20 as described in the Examples.

FIGS. 3A-3B depict histograms of particle size distribution for a formulation of the disclosure containing 20 mM histidine-chloride buffer, 115 mM NaCl and 0.05% polysorbate-20 as described in the Examples.

### DETAILED DESCRIPTION

### Definitions

As used herein, “during storage,” refers to a formulation that once prepared, is not immediately used; rather, following its preparation, it is packaged for storage, either in a liquid form, in a frozen state, or in a dried form (for later reconstitution into a liquid form).

As used herein, “aggregate” refers to a physical interaction between protein molecules, which results in the formation of covalent or non-covalent dimers or oligomers, which may remain soluble, or form insoluble aggregates that precipitate out of solution. An “aggregate” also refers to degraded and/or fragmented therapeutic proteins, such as degraded and/or fragmented antibodies or antigen-binding fragments thereof as herein described.

As used herein, a “particle” refers to liquids, e.g., droplets.

As used herein, “aerosolization” refers to the production of an aerosol by the transformation of a formulation into small particles or droplets, e.g., by use of an aerosol delivery system, e.g. nebulizer, as described herein.

As used herein, “nebulize” and “nebulization” refer to the conversion of a liquid into a mist or fine spray by a nebulizer as described herein.

As used herein, a “pharmaceutical formulation” refers to formulations which are in such a form as to permit the biological activity of the active ingredients to be effective, and, therefore, may be administered to a subject for therapeutic use as described herein.

As used herein, the term “protein” may be used herein interchangeably with the term “polypeptide” and, as used herein, encompasses a peptide, a polypeptide, a protein, and a fusion protein. Proteins may be made by recombinant or synthetic methods.

As used herein, the terms “subject” and “patient” are used interchangeably. As used herein, the terms “subject” and “subjects” refer to an animal, typically a mammal including a non-primate (e.g., a cow, pig, horse, cat, dog, rat, and mouse) and a primate (e.g., a monkey, such as a cynomolgus monkey, chimpanzee, baboon and a human), and more typically a human.

As used herein, the terms “therapeutic agent” and “therapeutic agents” refer to any agent(s), such as a drug or protein including antibodies and antigen-binding fragments thereof as described herein that can be used in the prevention, treatment and/or management of one or more diseases and/or disorders.

As used herein, the term “therapeutically effective amount” refers to the amount of a therapeutic agent, which is sufficient to reduce the severity of one or more diseases and/or disorders.

As used herein, the term “excipient” refers to an inert substance which is commonly used as a diluent, vehicle, preservative, binder or stabilizing agent for drugs which imparts a beneficial physical property to a formulation, such as increased protein stability, increased protein solubility, and/or decreased viscosity. Examples of excipients include, but are not limited to, proteins (e.g., serum albumin), amino acids (e.g., aspartic acid, glutamic acid, lysine, arginine, glycine, histidine), surfactants (e.g., sodium dodecyl sulfate (SDS), polysorbates such as Tween 20 and Tween 80, poloxamers such as Pluronics, and other nonionic surfactants such as polyethylene glycol) (PEG)), saccharides (e.g., glucose, sucrose, maltose and trehalose), polyols (e.g., mannitol and sorbitol), fatty acids and phospholipids (e.g., alkyl sulfonates and caprylate). For additional information regarding excipients, see Remington's Pharmaceutical Sciences (by Joseph P. Remington, 18th ed., 1990, Mack Publishing Co., Easton, Pa.), which is incorporated by reference herein in its entirety.

As used herein, an “antibody” describes an immunoglobulin whether natural or partly or wholly synthetically produced. The term also encompasses “antigen-binding fragments” as described herein. The term encompasses polyclonal, monoclonal, monospecific monoclonal antibodies, multispecific antibodies such as bi-specific monoclonal antibodies or tri-specific monoclonal antibodies, isolated monoclonal antibodies, recombinant monoclonal antibodies, and isolated human or humanized monoclonal antibodies, the last mentioned described in further detail in U.S. Pat. Nos. 4,816,397 and 4,816,567, which are each incorporated by reference in its entirety. Immunoglobulin molecules can be of any class (e.g., IgG, IgE, IgM, IgD, IgA, and IgY), or subclass (e.g., IgG1, IgG2, IgG3, IgG4, IgA1, and IgA2). Preferred antibodies are of the IgG class. The term “antibody(ies)” includes a wild type immunoglobulin (Ig) molecule, generally comprising four full length polypeptide chains, two heavy (H) chains and two light (L) chains; including full length functional mutants, variants, or derivatives thereof, which retain the essential epitope binding features of an Ig molecule.

As used herein, an “antigen-binding fragment” refers to a molecule other than an intact antibody that comprises a portion of an intact antibody that binds the antigen to which the intact antibody binds. Examples of antibody-binding fragments include but are not limited to (i) a Fab fragment; (ii) a F(ab′)2 fragment; (iii) a heavy chain portion of a Fab (Fd) fragment, which comprises VH and CH1 domains; (iv) a single chain Fab (scFAb) which is described, for example, in U.S. Publication No. 2007/0274985 and is herein incorporated by reference in its entirety; (v) a Fab′-like fragment, which differs from a Fab fragment in that the Fab′-like fragment is slightly larger having more heavy chain and typically having one or more additional sulfhydryl groups on its heavy chain; (vi) a domain antibody (dAb) fragment, which comprises a single variable domain; (vii) a camelid antibody; (viii) a variable fragment (Fv) fragment, which comprises the VL and VH domains of a single arm of an antibody, (ix) a single chain Fv fragment (scFv) wherein a VH domain and a VL domain are linked by a linker that allows the two domains to associate to form an antigen binding site; (x) multivalent antibody fragments (scFv dimers, trimers and/or tetramers; (xi) a diabody, which is a bivalent, bispecific antibody in which VH and VL domains are expressed on a single polypeptide chain, but which uses a linker that is too short to allow for pairing between the two domains on the same chain, thereby forcing the domains to pair with the complementarity domains of another chain and creating two antigen binding sites; (xii) a linear antibody, which comprises a pair of tandem Fv segments (VH-CH1-VH-CH1) which, together with complementarity light chain polypeptides, form a pair of antigen binding regions; (xiii) a minibody, which is a bivalent molecule comprising an scFv fused to constant immunoglobulin domains, CH3 or CH4, wherein the constant CH3 or CH4 domains serve as dimerization domains; and (ix) other non-full length portions of heavy and/or light chains, or mutants, variants, or derivatives thereof, alone or in any combination.

As used herein, “monoclonal antibody” refers to an antibody obtained from a population of substantially homogeneous antibodies, i.e., the individual antibodies comprising the population are identical and/or bind the same epitope, except for possible variant antibodies, e.g., containing naturally occurring mutations or arising during production of a monoclonal antibody preparation, such variants generally being present in minor amounts. A monoclonal antibody thus typically displays a single binding affinity for any antigen with which it immunoreacts. In contrast to polyclonal antibody preparations, which typically include different antibodies directed against different determinants (epitopes), each monoclonal antibody of a monoclonal antibody preparation is directed against a single determinant on an antigen. The modifier “monoclonal” indicates the character of the antibody as being obtained from a substantially homogeneous population of antibodies and is not to be construed as requiring production of the antibody by any particular method. For example, the monoclonal antibodies to be used in accordance with the present disclosure may be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies described, for example, in WO 2015/290097, which is herein incorporated by reference in its entirety.

As used herein, “Fab fragment” refers to an antibody fragment comprising a light chain fragment comprising a VL domain and a constant domain of a light chain (CL), and a VH domain and a first constant domain (CH1) of a heavy chain. Fab and F(ab′)2 portions of antibody molecules may be prepared by the proteolytic reaction of papain and pepsin, respectively, on substantially intact antibody molecules by methods that are well-known or may be prepared synthetically or recombinantly. Fab′ antibody molecule portions are also well-known and may be produced from F(ab′)₂ portions followed by reduction of the disulfide bonds linking the two heavy chain portions as with mercaptoethanol, and followed by alkylation of the resulting protein mercaptan with a reagent such as iodoacetamide.

As used herein, “Fc domain” refers to a C-terminal region of an immunoglobulin heavy chain that contains at least a portion of the constant region. For example, in natural IgG antibodies, the Fc domain is composed of two identical protein fragments, derived from the second and third constant domains of the antibody's two heavy chains.

As used herein, “variable region” or “variable domain” refers to the domain of an antibody heavy or light chain that is involved in binding the antibody to antigen. The variable domains of the heavy chain and light chain (VH and VL, respectively) of a native antibody generally have similar structures, with each domain comprising four conserved framework regions (FRs) and three hypervariable regions (HVRs). A single VH or VL domain may be sufficient to confer antigen-binding specificity. Furthermore, antibodies that bind a particular antigen may be isolated using a VH or VL domain from an antibody that binds the antigen to screen a library of complementary VL or VH domains, respectively.

As used herein, “epitope” refers to a region of an antigen (e.g., polypeptide) that is bound by the antigen-binding site of an antibody. In certain embodiments, an epitope determinant includes chemically active surface groupings of molecules such as amino acids, sugar side chains, phosphoryl, or sulfonyl, and, in certain embodiments, may have specific three-dimensional structural characteristics, and or specific charge characteristics.

As used herein, “percent amino acid sequence identity” refers to the percentage of amino acid residues in a candidate sequence that are identical with the amino acid residues in the reference polypeptide sequence, such as an antibody comprising a heavy chain and a light chain, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for example, using publicly available software such as BLAST or software available commercially, for example from DNASTAR. Two or more polypeptide sequences can be anywhere from 0-100% identical, or any integer value there between. In the context of the present disclosure, two polypeptides are “substantially identical” when at least 80% of the amino acid residues (such as at least about 85%, at least about 90%, at least about 92.5%, at least about 95%, at least about 98%, or at least about 99%) are identical. The term “percent (%) amino acid sequence identity” as described herein applies to peptides as well. Thus, the term “substantially identical” will encompass mutated, truncated, fused, or otherwise sequence-modified variants of antibodies, antigen-binding fragments thereof, as well as polypeptides, such as antibodies, with substantial sequence identity (e.g., at least 80%, at least 85%, at least 90%, at least 92.5%, at least 95%, at least 98%, or at least 99% identity as measured for example by one or more methods referenced above) as compared to the reference (wild type or other intact) polypeptide, such as an antibody.

As used herein, two amino acid sequences, such as two antibodies, e.g., comprising a heavy chain, are “substantially homologous” when at least about 80% of the amino acid residues (such as at least about 85%, at least about 90%, at least about 92.5%, at least about 95%, at least about 98%, or at least about 99%) are identical, or represent conservative substitutions. The sequences of the polypeptides, such as the antibodies of the present disclosure, are substantially homologous when one or more, such as up to 10%, up to 15%, or up to 20% of the amino acids of the polypeptide, such as the antibodies described herein, are substituted with a similar or conservative amino acid substitution, and wherein the resulting peptides have at least one activity (e.g., ability to bind a specific epitope, neutralizing activity) of the reference polypeptide, such as an activity of an antibody described herein.

As used herein, a “conservative amino acid substitution” refers to one in which the amino acid residue is replaced with an amino acid residue having a side chain with a similar charge. Families of amino acid residues having side chains with similar charges have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine).

As used herein, “preventing” or “prevention” refers to a reduction in risk of acquiring or developing a disease or disorder (i.e., causing at least one of the clinical symptoms of the disease not to develop) in a subject that may be exposed to a disease causing agent, or predisposed to the disease in advance of disease onset.

As used herein, “prophylaxis” is related to and encompassed in the term “prevention” and refers to a measure or procedure, the purpose of which is to prevent, rather than to treat or cure a disease.

As used herein “intranasal” refers to administering, e.g., a formulation of the disclosure, within or via the nose or nasal structures or airway delivery, for example by inhalation. The term intranasal as used herein is not intended to be limited to or to imply limitation to administration directly or specifically or solely via the nose or nasal cavity, particularly in serving to exclude other means of administration whereby drug, agent, antibody, fragment, composition is delivered or otherwise provided to, deposited in or at or otherwise distributed to the respiratory tract.

As used herein, “inhalation” refers to taking in, particularly in the context of taking in or administering/being administered an agent or compound, including an antibody, or a composition comprising such, whereby the agent, compound, antibody, including as comprised in the formulation, is delivered to all or part of the respiratory tract. Inhalation may occur via the nose or via the mouth, or via direct administration to the lower respiratory tract as in intratracheal administration. Thus, inhalation may include nose only or primarily, intranasal, inhaling via the mouth, oral inhalation, intratracheal inhalation, intratracheal instillation. Thus inhalation provides for and contemplates any means of administration to the respiratory tract exclusively, specifically or preferentially, including the upper and/or lower respiratory tract, whereby drug, agent, composition or antibody reaches or is deposited at or in the respiratory tract exclusively, specifically or preferentially, including the upper and/or lower respiratory tract.

As used herein, the term “treating” or “treatment” refers to any process, action, application, therapy, or the like, wherein a subject, such as a human being, is subjected to medical aid with the object of curing a disorder, eradicating a pathogen, or improving the subject's condition, directly or indirectly. Treatment also refers to reducing incidence, alleviating symptoms, eliminating recurrence, preventing recurrence, preventing incidence, reducing the risk of incidence, improving symptoms, improving prognosis, or combinations thereof. “Treatment” may further encompass reducing the propagation of a virus in a subject and thereby controlling or reducing a viral infection, such as an influenza viral infection in a subject or viral contamination of an organ, tissue, or environment.

### Formulations

The present disclosure is directed to a therapeutic protein formulation, typically for respiratory tract delivery. In some embodiments, the present therapeutic protein formulation (also referred to herein as a “formulation”, a “composition of matter” or a “composition”) is a liquid formulation, more typically an aqueous formulation. As used herein, an “aqueous formulation” is a formulation in which the solvent is water. In some embodiments, the present formulation is a lyophilized formulation, a freeze-dried formulation or a spray-dried formulation, which may be reconstituted as a liquid, e.g., an aqueous formulation, prior to administration to a subject.

In some embodiments, the present formulation is useful for respiratory tract delivery via aerosolization. Accordingly, the present formulations have a viscosity that is compatible with aerosolization. Typically, the present formulation exhibits a dynamic viscosity in the range of about 0.8 mPa s to about 17.0 mPa s at a temperature of about 20° C., such as about 2 to 8 mPa s, such as about 3 to 7 mPa s, such as about 3 to 4 mPa s.

Generally, high concentrations of therapeutic protein are used in the present formulation. Although high doses of therapeutic protein are typical in the disclosure, the volume to be aerosolized may be minimized in order to keep the aerosolization time as short as possible, typically to promote patient compliance. Typically, the concentration of therapeutic protein in the present formulation ranges from about 5 mg/mL to about 150 mg/mL, such as from about 10 mg/mL to about 120 mg/mL, such as from about 15 mg/mL to about 100 mg/mL, such as from about 30 mg/mL to about 70 mg/mL, such as from about 40 mg/mL to about 60 mg/mL. More typically, the concentration of therapeutic protein in the present formulation is about 50 mg/mL.

Generally, the present therapeutic protein formulation is a stable formulation. A “stable” formulation is one in which the therapeutic protein in the formulation essentially retains its physical stability and/or chemical stability and/or biological activity upon storage, including storage in a reservoir of an aerosolization device, such as a nebulizer as described herein or during aerosolization. A protein “retains its physical stability” in a formulation if it shows little to no change in aggregation, precipitation and/or denaturation as observed by, for example, visual examination of color and/or clarity, or as measured by UV light scattering (measures visible aggregates) or size exclusion chromatography (SEC).

A protein “retains its chemical stability” in a formulation if the protein is not degraded. Chemical stability can be assessed by detecting and quantifying chemically altered forms of the protein. Chemical alteration may involve size modification (e.g. clipping), which can be evaluated, for example, using SEC, sodium dodecyl sulfate-polyacrylamide gel electrophoresis (SDS-PAGE) and/or matrix-assisted laser desorption ionization/time-of-flight mass spectrometry (MALDI/TOF MS). Other types of chemical alteration include charge alteration (e.g. occurring as a result of deamidation), which can be evaluated by e.g., ion-exchange chromatography.

The therapeutic protein of the present formulation, which typically comprises one or more antibodies as described herein, “retains its biological activity” in a formulation if the biological activity of e.g., the antibody, at a given time, such as before storage or aerosolization, is within about 10% of its biological activity exhibited at the time the formulation was prepared as determined, e.g., in an antigen-binding assay, such as an enzyme-linked immunosorbent assay (ELISA) assay.

The term “stable” with respect to the formulation of the present disclosure during storage or aerosolization is understood to mean that the therapeutic protein of the present formulation does not lose more than 15%, or more typically 10%, or even more typically 5%, and yet even more typically 3% of its biological activity during storage relative to its activity at the beginning of storage or prior to aerosolization.

In some embodiments, the present formulation results in a reduction in the amount of aggregation of the therapeutic protein during storage, including storage in a reservoir of an aerosolization device, such as a nebulizer or during aerosolization in comparison to the amount of aggregation of the therapeutic protein during storage or aerosolization in the absence of the stabilizing agents as herein described.

Aggregates may be formed, for example, because of exposure to elevated temperatures. By “elevated temperature” is meant any temperature above the temperature at which the formulation of the disclosure comprising the present therapeutic antibodies is normally stored. The normal storage temperature is between about −70° C. and 8° C., typically about −20° C., or about 4° C. and 8° C., more typically between about 4° C. and 6° C. and even more typically at a temperature of about 4° C. In some embodiments, the present therapeutic protein formulation provided herein is also stabilized at room temperature (i.e., between 20° C. and 25° C.).

Further causes for the formation of aggregates during storage are due to the inherent tendency of therapeutic proteins, such as the therapeutic proteins described herein, to form aggregates. Without being bound by theory, it is assumed that aggregate formation of the present therapeutic proteins may lead to a loss of activity.

Aggregate formation during storage or aerosolization can be assessed by various analytical and/or immunological methods known in the art including but not limited to e.g. size exclusion chromatography (SE-HPLC), subvisible particle counting, analytical ultracentrifugation (AUC), dynamic light scattering (DLS), static light scattering (SLS), elastic light scattering, OD320/OD280, Fourier Transform Infrared Spectroscopy (FTIR), circular dichroism (CD), urea-induced protein unfolding techniques, intrinsic tryptophan fluorescence and/or differential scanning calorimetry techniques. Typically, SE-HPLC is used to assess the molecular size distribution and the relative amounts of the present therapeutic proteins and impurities during storage. SE-HPLC methods are known to the skilled person.

In some embodiments, the present therapeutic proteins include no more than 20%, no more than 10%, no more than 7%, no more than 6%, no more than 5%, no more than 4%, no more than 3%, no more than 2%, no more than 1%, or no more than 0.5% aggregation by weight of protein during storage in comparison to the aggregation by weight of the therapeutic protein at the beginning of storage or aerosolization.

In some embodiments, the present therapeutic protein formulation provided herein is stored between about 4° C. and 8° C. for an extended period of time. In some embodiments, the present therapeutic protein formulation is stable when stored between about 4° C. and 8° C. for at least about 1 month. In some embodiments, the present formulation is stable when stored between about 4° C. and 8° C. for at least about 3 months. In yet other embodiments, the present formulation is stable when stored between about 4° C. and 8° C. for at least 6 months, such as at least one year, such as at least two years.

In some embodiments, the present therapeutic protein formulation provided herein is stored between about −70° C. and −20° C. for an extended period of time. In some embodiments, the present therapeutic protein formulation is stable when stored between about −70° C. and −20° C. for at least about 1 month. In some embodiments, the present formulation is stable when stored between about −70° C. and −20° C. for at least about 3 months. In yet other embodiments, the present formulation is stable when stored between about −70° C. and −20° C. for at least 6 months, such as at least one year, such as at least two years.

In some embodiments, the present therapeutic protein of the formulation of the disclosure comprises a stabilizing agent, which comprises a charged amino acid, having a net charge at a pH between about 5.0 and 8.0, such as about 6.0 to about 6.5, such as about 6.0 in solution. The term “about” when used in the context of pH value/range refers to a numeric value having a range of +/−25% around the cited value. Without being limited by theory, the presence of the charged amino acids allows for the preparation of highly concentrated therapeutic protein formulations as described herein. In some embodiments, the charged amino acid is arginine, glutamate or histidine. Typically, the amino acid is histidine.

The term “net charge”, in reference to an amino acid as used herein, means that positive and negative charges on the surface of the amino acid are not zero. The net charge depends on pH. At a specific pH, the positive and negative charges will be balanced and the net charge will be zero, i.e., the isoelectric point.

Typically, the stabilizing agent comprises a histidine buffer. As used herein, a “buffer” refers to a buffered solution that resists changes in pH by the action of its acid-base conjugate components. As used herein, a “histidine buffer” is a buffer comprising histidine ions. Examples of histidine buffers include histidine chloride, histidine acetate, histidine phosphate and histidine sulfate. In some embodiments, the histidine buffer minimizes irritation to the lungs. In some embodiments, the histidine buffer excludes histidine acetate since this buffer may irritate the lungs. The most typical histidine buffer in the present formulation is histidine chloride. In a typical embodiment, the histidine chloride buffer is prepared by titrating L-histidine (free base, solid) with hydrochloric acid (liquid) or titrating Histidine buffer with Histidine chloride buffer solution to a predetermined pH. Typically, the histidine buffer or histidine-chloride buffer is at a pH of about 5.5 to about 7.5, typically a pH of about 6.0 to about 6.5, most typically about 6.0.

It is to be understood that the pH can be adjusted as necessary to maximize stability and solubility of the therapeutic protein in a particular aqueous formulation. The pH value of the present formulations may be adjusted by the addition of acidic agents or basic agents. The pH may be raised, or made more alkaline, by addition of an alkaline agent such as sodium hydroxide, ammonium hydroxide, potassium hydroxide, sodium carbonate, sodium bicarbonate or combinations thereof. Suitable acids for use as pH adjusting agents include, for example, hydrochloric acid, phosphoric acid, phosphorous acid, citric acid, glycolic acid, lactic acid, acetic acid, benzoic acid, malic acid, oxalic acid, tartaric acid, succinic acid, glutaric acid, valeric acid and the like. Typically, hydrochloric acid is used to adjust the pH of the present formulation if needed.

In typical embodiments, histidine is present in the formulation at a concentration ranging from 5 mM to 30 mM, such as between 10 mM and 25 mM, such as between 15 mM and 25 mM. In some embodiments, the concentration of histidine in the present formulation is 5.0±0.5 mM, 10.0±1 mM, 15.0±1.5 mM, 20±2 mM, 25±2.5 mM, 30±5 mM or lower. In other embodiments, the concentration of histidine in the formulation is 5.0±0.5 mM, 10.0±1 mM, 15.0±1.5 mM, 20±2 mM, 25±2.5 mM, 30±5 mM or higher. More typically, the concentration of histidine in the formulation is 20.0±0.5 mM, 20.0±1 mM, 20±1.5 mM, 20±2 mM, 20±2.5 mM or 20±5 mM. Even more typically, the concentration of histidine in the formulation is 20 mM.

It will be understood by one skilled in the art that the present therapeutic formulation may be isotonic or slightly hypotonic with human blood, i.e. the present therapeutic formulation of the disclosure has essentially the same or a slightly lower osmotic pressure as human blood. Such isotonic or slightly hypotonic formulation generally has an osmotic pressure from about 240 mOSm/kg to about 320 mOSm/kg, such as about 240 mOSm/kg or higher, 250 mOSm/kg or higher or 260 mOSm/kg or higher. Osmotic pressure can be measured, for example, using a vapor pressure or ice-freezing type osmometer.

Tonicity of the present formulation can be adjusted by the use of a tonicity adjuster. “Tonicity adjusters” are those pharmaceutically acceptable inert substances that can be added to the formulation to provide an isotonicity of a composition. A typical tonicity adjuster in the formulation of the disclosure is an inorganic salt. Without being limiting, inorganic salts for adjusting the osmolality of the composition of the disclosure include NaCl, KCl, CaCl₂, and MgCl₂, in particular NaCl. The concentration of inorganic salt may range from 10 mM to 200 mM, 10 mM to 150 mM, 50 mM to 150 mM, 100 mM to 150 mM, or 100 mM to 120 mM. In a specific aspect, the concentration of salt (typically NaCl) which may be included in the formulations of the disclosure may be about 10 mM, about 25 mM, about 50 mM, about 75 mM, about 100 mM, about 110 mM, about 115 mM, about 130 mM, about 150 mM, or about 200 mM.

In some embodiments, instead of or in addition to acting as a tonicity adjuster, NaCl may help reduce irritation in the respiratory tract, e.g. reduce coughing, and/or further stabilize the present therapeutic proteins. In some embodiments, the concentration of NaCl in the present formulation ranges from about 100 mM to about 200 mM. Typically, 115 mM of NaCl is used.

The formulations of the present disclosure may further contain one or more surfactants, typically non-ionic surfactants. Protein solutions, in particular antibody solutions, have a high surface tension at the air-water interface. In order to reduce this surface tension, proteins, such as antibodies, tend to aggregate at the air-water interface. A surfactant minimizes antibody aggregation at the air-water interface, thereby helping to maintain the biological activity of the antibody in solution or during aerosolization. When the formulation is lyophilized, the surfactant may also reduce the formation of particulates in the reconstituted formulation.

Certain exemplary non-ionic surfactants include fatty alcohol, polysorbates such as polysorbate 20, polysorbate 80, Triton X-100, polyoxypropylene-polyoxyethylene copolymer (PLURONIC®), and nonyl phenoxypolyethoxylethanol (NP-40). Other surfactants which can be used in the formulation of the disclosure include phosphoglycerides, such as phosphatidyl cholines (lecithin), such as the naturally occurring surfactant, dipalmitoyl phosphatidyl choline (DPPC). Other exemplary surfactants include diphosphatidyl glycerol (DPPG), hexadecanol, polyoxyethylene-9-lauryl ether, a surface active fatty acid, such as palmitic acid or oleic acid, sorbitan trioleate (Span 85), glycocholate, surfactin, a poloxamer, a sorbitan fatty acid ester such as sorbitan trioleate, tyloxapol and a phospholipid.

The concentration of the surfactant may range from between 0.001% and 1% (v:v) (typically between 0.001% and 0.1% (v:v), or between 0.01% and 0.1% (v:v) such as about 0.001% (v:v), 0.005% (v:v), 0.01% (v:v), 0.02% (v:v), 0.05% (v:v), 0.08% (v:v), 0.1% (v:v), 0.5% (v:v), or 1% (v:v) of the formulation, typically from about 0.04% to 0.08% (v:v)). In a specific embodiment, the surfactant is polysorbate 20 or polysorbate 80, which is at a concentration of 0.001% (v:v), 0.005% (v:v), 0.01% (v:v), 0.02% (v:v), 0.04%, 0.05% (v:v), 0.08% (v:v), 0.1% (v:v), 0.5% (v:v) or 1% (v:v) of the formulation, typically 0.04% to 0.08% (v:v).

An example of a typical formulation of the disclosure comprises 0.01% (v:v) polysorbate 80, 0.02% (v:v) polysorbate 80, 0.05% (v:v) polysorbate 80 or 0.08% (v:v) polysorbate 80. Typically, polysorbate 80, 0.02% (v:v) is used.

Another example of a typical formulation of the disclosure comprises 0.01% (v:v) polysorbate 20, 0.02% (v:v) polysorbate 20, 0.05% (v:v) polysorbate 20 or 0.08% (v:v) polysorbate 20. More typically, polysorbate 20, 0.02% (v:v) is used.

The present therapeutic protein formulation can also comprise further pharmaceutically acceptable excipients, which serve to optimize the characteristics of the formulation and/or the characteristics of the aerosol. Examples of such excipients include taste-masking agents, sweeteners, and flavors.

The formulation of the disclosure is typically prepared by combining, in addition to the therapeutic proteins as described herein, an amino acid, such as histidine, wherein the histidine has a net charge at a pH between about 5.5 and 8, in an aqueous carrier or combining the therapeutic proteins as described herein with an aqueous carrier further comprising a histidine buffer. Further, NaCl, or optionally a surfactant, pH adjusting agents and additional excipients can be added as needed. Persons having ordinary skill in the art will understand that the combining of the various components to be included in the formulation can be done in any appropriate order. For example, the buffer can be added first, middle or last and the surfactant can also be added first, middle or last. It is also to be understood by one of ordinary skill in the art that some of these chemicals can be incompatible in certain combinations, and accordingly, are easily substituted with different chemicals that have similar properties but are compatible in the relevant mixture.

### Therapeutic Proteins

In some embodiments, the one or more therapeutic proteins of the present formulation comprise an antibody for use in treating influenza. As used herein “influenza” encompasses influenza viruses that are circulating in the human population including influenza A, influenza B and influenza C. Typically, human influenza A and B viruses cause seasonal epidemics of disease. Influenza type C infections typically cause a mild respiratory illness and do not cause epidemics.

As used herein, “influenza A” encompasses any of the virus subtypes as determined by the type of hemagglutinin (H) and/or the type of neuraminidase (N) proteins on the surface of the virus, e.g., subtypes H1N1 and H3N2. As used herein, “influenza type B” encompasses any of the different lineages of influenza type B including the Yamagata and/or Victoria lineages.

Typically, the present antibodies are directed against influenza A and/or influenza B, such as influenza A H1 and H3 subtypes and the Yamagata and Victoria lineages of influenza B. Accordingly, in some embodiments the present formulations may comprise two, such as three, or more therapeutic proteins comprising the antibodies described herein.

In some embodiments, the antibodies directed against influenza A and/or B are neutralizing antibodies. Antibody-mediated neutralization of a virus can be tested in any conventional neutralization assay known in the art. Examples of suitable neutralization assays include conventional neutralization assays based on the inhibition of virus cytopathic effect (CPE) on cells in culture. For example, influenza neutralization may be tested by reducing or blocking formation of CPE in Madin-Darby Canine Kidney (MDCK) cells infected with influenza. The virus and putative neutralizing agent may be premixed before addition to cells, followed by measuring blocking of virus entry.

Conversely, some neutralization assays can detect blocking of virus egress, as in the case of neuraminidase inhibitors like TAMIFLU®. For example, in some embodiments, an antibody is provided that may not appear to be a neutralizing antibody in a conventional in vitro neutralization assay, but it exhibits egress inhibition neutralization. Thus, a “neutralizing” antibody as used herein refers to an antibody exhibiting neutralization in a conventional in vitro neutralization assay and/or an antibody that exhibits egress inhibition. An antibody that is a virus egress inhibitor is neutralizing in the sense that it inhibits propagation of an influenza infection.

In some embodiments, the antibodies of the present disclosure, such as the neutralizing antibodies of the disclosure, are monoclonal antibodies. Methods for making monoclonal antibodies by hybridomas or other means and approaches are well known. See, for example, Niman et al, Proc. Natl. Acad. Sci. USA, 1983, 80:4949-4953, which is herein incorporated by reference in its entirety. Typically, a virus, viral protein, or a peptide analog is used either alone or conjugated to an immunogenic carrier, as the immunogen for producing monoclonal antibodies. The hybridomas are screened for the ability to produce an antibody that immunoreacts with the virus, protein or peptide analog.

The antibody, such as a monoclonal antibody, of the present disclosure, such as a neutralizing monoclonal antibody, may neutralize any of the hemagglutinin (H) and/or neuraminidase (N) subtypes, such as the H1N1 and H3N2 viruses. In some embodiments, the antibody neutralizes a human influenza A virus from Group 1 hemagglutinin subtypes including H1, H2, H5, H6, H8, H9, H11, H13 and H16, typically, the H1 subtype. In some embodiments, the antibody neutralizes a human influenza A virus from a Group 2 hemagglutinin subtypes, including H3, H4, H7, H10, H15 and H14, typically, the H3 subtype.

The antibody, such as a monoclonal antibody, of the present disclosure, such as a neutralizing monoclonal antibody, may neutralize human influenza B viruses, such as those from the Yamagata and/or Victoria lineages. The anti-influenza antibody of the present disclosure may be strain specific or non-specific or pan-specific.

More particularly, antibodies suitable for use in the present formulations and methods of the disclosure include those described in WO 2015/120097 and WO 2014/152841, each of which is herein incorporated by reference in its entirety. For example, in some embodiments, the present formulation includes a therapeutic protein comprising a Group 1 monoclonal antibody such as TRL053/Mab53, a therapeutic protein comprising a Group 2 antibody, such as monoclonal TRL579/Mab579, or a therapeutic protein comprising an anti-influenza B antibody selected from at least one of the monoclonal antibodies TRL809, TRL812, TRL813, TRL832, TRL841, TRL842, TRL845, TRL846, TRL847, TRL848, TRL849, TRL854 and TRL856. In particular embodiments, the anti-influenza B antibody comprises TRL849. In some embodiments, the present formulation comprises humanized or chimerized versions of the foregoing antibodies.

In some embodiments, the present antibodies may be directed against more than one influenza strain or subtype. For example, TRL053/Mab53 is effective against influenza A H1, H9, H7 and H5, which are subtypes of Group 1 and 2. TRL579/Mab579 is effective against H3 and H7 subtypes. Thus, while the predominant strains of circulating influenza strains include H1, H3 and B types, combinations having efficacy against additional strains and subtypes, including subtypes which may arise and emerge in a new or single flu season, can be generated.

As is well known, the specificity of monoclonal antibodies is essentially determined by the complementarity-determining regions (CDRs) that are present in the variable regions of the light and heavy chains. Accordingly, in some embodiments, a therapeutic protein formulation is provided, which comprises a therapeutic protein comprising light and/or heavy chain CDRs.

In some embodiments, the present therapeutic formulation comprises one or more anti-influenza monoclonal antibodies comprising a heavy and a light chain, wherein the heavy chain comprises: (a1) the TRL053 heavy chain CDR sequences HCDR1/HCDR2/HCDR3 (SEQ ID NOS: 1, 2, 3, respectively). In other embodiments, the one or more anti-influenza monoclonal antibodies binds to the same epitope as the antibody of (a1) and comprises a heavy and a light chain, wherein the heavy chain comprises: (a2) a polypeptide having at least 80%, such as at least 85%, such as at least 90%, such as at least 95%, such as at least 98%, or such as at least 99% sequence identity with the TRL053 heavy chain CDR sequences HCDR1/HCDR2/HCDR3 (SEQ ID NOS: 1, 2, 3, respectively).

In addition or alternatively to (a1) and/or (a2), the present therapeutic formulation comprises one or more anti-influenza monoclonal antibodies comprising a heavy and a light chain, wherein the heavy chain comprises: (b1) the TRL579 heavy chain CDR sequences HCDR1/HCDR2/HCDR3 (SEQ ID NOS: 7, 8, 9, respectively) and/or the one or more anti-influenza monoclonal antibodies binds to the same epitope as the antibody of (b1) and comprises a heavy and a light chain, wherein the heavy chain comprises: (b2) a polypeptide having at least 80%, such as at least 85%, such as at least 90%, such as at least 95%, such as at least 98%, or such as at least 99% sequence identity with the TRL579 heavy chain CDR sequences HCDR1/HCDR2/HCDR3 (SEQ ID NOS: 7, 8, 9, respectively).

In addition or alternatively to (a1), (a2), (b1) or (b2), the present therapeutic formulation comprises one or more anti-influenza monoclonal antibodies comprising a heavy and a light chain, wherein the heavy chain comprises: (c1) the heavy chain CDR sequences, HCDR1/HCDR2/HCDR3, selected from: (i) SEQ ID NOS: 13, 14, and 15, respectively (TRL809); (ii) SEQ ID NOS: 19, 20, and 21, respectively (TRL812); (iii) SEQ ID NOS: 25, 26, and 27, respectively (TRL813); (iv) SEQ ID NOS: 31, 32, and 33, respectively (TRL832); (v) SEQ ID NOS: 37, 38, and 39, respectively (TRL841); (vi) SEQ ID NOS: 43, 44, and 45, respectively (TRL842); (vii) SEQ ID NOS: 49, 50, and 51, respectively (TRL845); (viii) SEQ ID NOS: 55, 56, and 57, respectively (TRL846); (ix) SEQ ID NOS: 61, 62, and 63, respectively (TRL847); (x) SEQ ID NOS: 67, 68, and 69, respectively (TRL848); (xi) SEQ ID NOS: 73, 74, and 75, respectively (TRL849); (xii) SEQ ID NOS: 79; 80, and 81, respectively (TRL854); and (xiii) SEQ ID NOS: 85, 86, and 87, respectively and (xiv) SEQ ID NOS: 88, 89, 90, respectively (TRL856), and/or wherein the one or more anti-influenza monoclonal antibodies binds to the same epitope as the antibody of (c1) and comprises a heavy and a light chain, wherein the heavy chain comprises: a polypeptide having at least 80%, such as at least 85%, such as at least 90%, such as at least 95%, such as at least 98%, or such as at least 99% sequence identity with the heavy chain CDR sequences, HCDR1/HCDR2/HCDR3, selected from: i) SEQ ID NOS: 13, 14, and 15, respectively (TRL809); (ii) SEQ ID NOS: 19, 20, and 21, respectively (TRL812); (iii) SEQ ID NOS: 25, 26, and 27, respectively (TRL813); (iv) SEQ ID NOS: 31, 32, and 33, respectively (TRL832); (v) SEQ ID NOS: 37, 38, and 39, respectively (TRL841); (vi) SEQ ID NOS: 43, 44, and 45, respectively (TRL842); (vii) SEQ ID NOS: 49, 50, and 51, respectively (TRL845); (viii) SEQ ID NOS: 55, 56, and 57, respectively (TRL846); (ix) SEQ ID NOS: 61, 62, and 63, respectively (TRL847); (x) SEQ ID NOS: 67, 68, and 69, respectively (TRL848); (xi) SEQ ID NOS: 73, 74, and 75, respectively (TRL849); (xii) SEQ ID NOS: 79; 80, and 81, respectively (TRL854); and (xiii) SEQ ID NOS: 85, 86, and 87, respectively and (xiv) SEQ ID NOS: 88, 89, 90, respectively (TRL856).

Typically, the present formulation includes at least three anti-influenza monoclonal antibodies comprising a heavy and a light chain, wherein

(i) the heavy chain of the first antibody comprises: the TRL053 heavy chain CDR sequences HCDR1/HCDR2/HCDR3 (SEQ ID NOS: 1, 2, 3, respectively);

(ii) the heavy chain of the second antibody comprises: the TRL579 heavy chain CDR sequences HCDR1/HCDR2/HCDR3 (SEQ ID NOS: 7, 8, 9, respectively); and

(iii) the heavy chain of the third antibody comprises: the TRL849 heavy chain CDR sequences HCDR1/HCDR2/HCDR3 (SEQ ID NOS: 73, 74, and 75, respectively).

In some embodiments, the present therapeutic formulation comprises one or more anti-influenza monoclonal antibodies comprising a heavy and a light chain, wherein the heavy and the light chain comprise: (a1) the TRL053 heavy chain CDR sequences HCDR1/HCDR2/HCDR3 (SEQ ID NOS: 1, 2, 3, respectively) and the TRL053 light chain CDR sequences LCDR1/LCDR2/LCDR3 (SEQ ID NOS: 4, 5, 6, respectively); and/or wherein the one or more anti-influenza monoclonal antibodies binds to the same epitope as the antibody of (a1) and comprises a heavy and a light chain, wherein the heavy chain and light chain comprise: (a2) a polypeptide having at least 80%, such as at least 85%, such as at least 90%, such as at least 95%, such as at least 98%, or such as at least 99% sequence identity with the TRL053 heavy chain CDR sequences HCDR1/HCDR2/HCDR3 (SEQ ID NOS: 1, 2, 3, respectively) and the TRL053 light chain CDR sequences LCDR1/LCDR2/LCDR3 (SEQ ID NOS: 4, 5, 6, respectively).

In addition or alternatively to (a1) and/or (a2), above, the present therapeutic formulation comprises one or more anti-influenza monoclonal antibodies comprising a heavy and a light chain, wherein the heavy and the light chain comprise: (b1) the TRL579 heavy chain CDR sequences HCDR1/HCDR2/HCDR3 (SEQ ID NOS: 7, 8, 9, respectively) and the TRL579 light chain CDR sequences LCDR1/LCDR2/LCDR3 (SEQ ID NOS: 10, 11, 12, respectively) and/or wherein the one or more anti-influenza monoclonal antibodies binds to the same epitope as the antibody of (b1) and comprises a heavy and a light chain, wherein the heavy and the light chain comprise: (b2) a polypeptide having at least 80%, such as at least 85%, such as at least 90%, such as at least 95%, such as at least 98%, or such as at least 99% sequence identity with the TRL579 heavy chain CDR sequences HCDR1/HCDR2/HCDR3 (SEQ ID NOS: 7, 8, 9, respectively) and the TRL579 light chain CDR sequences LCDR1/LCDR2/LCDR3 (SEQ ID NOS: 10, 11, 12, respectively).

In addition or alternatively to (a1), (a2), (b1) or (b2), the present therapeutic formulation comprises one or more anti-influenza monoclonal antibodies comprising a heavy and a light chain, wherein the heavy and the light chain comprise: (c1) the heavy and light chain CDR sequences, HCDR1/HCDR2/HCDR3, and LCDR1/LCDR2/LCDR3, respectively, selected from: (i) SEQ ID NOS: 13, 14, 15 and SEQ ID NOS: 16, 17, 18 (TRL809); (ii) SEQ ID NOS: 19, 20, 21 and SEQ ID NOS: 22, 23, 24 (TRL812); (iii) SEQ ID NOS: 25, 26, 27 and SEQ ID NOS: 28, 29, 30 (TRL813); (iv) SEQ ID NOS: 31, 32, 33 and SEQ ID NOS: 34, 35, 36 (TRL832); (v) SEQ ID NOS: 37, 38, 39 and SEQ ID NOS: 40, 41, 42 (TRL841); (vi) SEQ ID NOS: 43, 44, 45 and SEQ ID NOS: 46, 47, 48 (TRL842); (vii) SEQ ID NOS: 49, 50, 51 and SEQ ID NOS: 52, 53, 54 (TRL845); (viii) SEQ ID NOS: 55, 56, 57 and SEQ ID NOS: 58, 59, 60 (TRL846); (ix) SEQ ID NOS: 61, 62, 63 and SEQ ID NOS: 64, 65, 66 (TRL847); (x) SEQ ID NOS: 67, 68, 69 and SEQ ID NOS: 70, 71, 72 (TRL848); (xi) SEQ ID NOS: 73, 74, 75 and SEQ ID NOS: 76, 77, 78 (TRL849); (xii) SEQ ID NOS: 79; 80, 81 and SEQ ID NOS: 82, 83, 84 (TRL854) and (xiii) SEQ ID NOS: 85, 86, 87, and SEQ ID NOS: 88, 89, 90 (TRL856) and/or wherein the one or more anti-influenza monoclonal antibodies binds to the same epitope as the antibody of (c1) and comprises a heavy and a light chain, wherein the heavy and the light chain comprise: (c2) a polypeptide having at least 80%, such as at least 85%, such as at least 90%, such as at least 95%, such as at least 98%, or such as at least 99% sequence identity with the heavy and light chain CDR sequences, HCDR1/HCDR2/HCDR3 and LCDR1/LCDR2/LCDR3, respectively, selected from: (i) SEQ ID NOS: 13, 14, 15 and SEQ ID NOS: 16, 17, 18 (TRL809); (ii) SEQ ID NOS: 19, 20, 21 and SEQ ID NOS: 22, 23, 24 (TRL812); (iii) SEQ ID NOS: 25, 26, 27 and SEQ ID NOS: 28, 29, 30 (TRL813); (iv) SEQ ID NOS: 31, 32, 33 and SEQ ID NOS: 34, 35, 36 (TRL832); (v) SEQ ID NOS: 37, 38, 39 and SEQ ID NOS: 40, 41, 42 (TRL841); (vi) SEQ ID NOS: 43, 44, 45 and SEQ ID NOS: 46, 47, 48 (TRL842); (vii) SEQ ID NOS: 49, 50, 51 and SEQ ID NOS: 52, 53, 54 (TRL845); (viii) SEQ ID NOS: 55, 56, 57 and SEQ ID NOS: 58, 59, 60 (TRL846); (ix) SEQ ID NOS: 61, 62, 63 and SEQ ID NOS: 64, 65, 66 (TRL847); (x) SEQ ID NOS: 67, 68, 69 and SEQ ID NOS: 70, 71, 72 (TRL848); (xi) SEQ ID NOS: 73, 74, 75 and SEQ ID NOS: 76, 77, 78 (TRL849); (xii) SEQ ID NOS: 79; 80, 81 and SEQ ID NOS: 82, 83, 84 (TRL854) and (xiii) SEQ ID NOS: 85, 86, 87, and SEQ ID NOS: 88, 89, 90 (TRL856).

Typically, the present formulation includes at least three anti-influenza monoclonal antibodies comprising a heavy and a light chain, wherein the heavy and the light chain of the first antibody comprises:

(i) the TRL053 heavy chain CDR sequences HCDR1/HCDR2/HCDR3 (SEQ ID NOS: 1, 2, 3, respectively) and the TRL053 light chain CDR sequences LCDR1/LCDR2/LCDR3 (SEQ ID NOS: 4, 5, 6, respectively);

(ii) the heavy and light chain of the second antibody comprises: the TRL579 heavy chain CDR sequences HCDR1/HCDR2/HCDR3 (SEQ ID NOS: 7, 8, 9, respectively) and the TRL579 light chain CDR sequences LCDR1/LCDR2/LCDR3 (SEQ ID NOS: 10, 11, 12, respectively); and

(iii) the heavy and light chain of the third antibody comprise: the TRL849 heavy chain

CDR sequences, HCDR1/HCDR2/HCDR3 (SEQ ID NOS: 73, 74, and 75, respectively) and the TRL849 light chain CDR sequences (LCDR1/LCDR2/LCDR3 of SEQ ID NOS: 76, 77, 78, respectively).

In some embodiments, the monoclonal antibodies in the present formulation are all of the same IgG subtype and have identical or near identical constant region sequences. In a particular aspect, all antibodies in the combination are IgG1 antibodies. In some embodiments, the antibodies are typically designed and expressed with similar or comparable constant region sequences and are typically of the same IgG, selected from human IgG1, IgG2, IgG2, IgG3, or IgG4. Modified Fc sequences to provide longer half-life in circulation are also known in the art.

In some embodiments, the present antibodies comprise human heavy and light chain constant regions as are known in the art. In some embodiments, the present anti-influenza B antibodies comprise a human IgG1 constant region amino acid sequence. In some embodiments, anti-influenza B antibodies are provided comprising a human IgG1 constant region amino acid sequence of SEQ ID NO: 93. In some embodiments, the present anti-influenza B antibodies comprise a human light chain kappa constant region of SEQ ID NO: 91 or a human light chain lambda constant region of SEQ ID NO: 92.

In some embodiments, the present therapeutic formulation comprises one or more anti-influenza monoclonal antibodies comprising a heavy and a light chain, wherein the heavy chain comprises: (a1) the heavy chain amino acid sequence of TRL053 (SEQ ID NO: 94). In other embodiments, the one or more anti-influenza monoclonal antibodies binds to the same epitope as the antibody of (a1) and comprises a heavy and a light chain, wherein the heavy chain comprises: (a2) a polypeptide having at least 80%, such as at least 85%, such as at least 90%, such as at least 95%, such as at least 98%, or such as at least 99% sequence identity with the amino acid sequence of TRL053 (SEQ ID NO: 94). In certain embodiments of (a2), any amino acid substitutions, additions, or deletions in the heavy chain amino acid sequence of TRL053 (SEQ ID NO: 94) are located in the framework regions. In certain embodiments of (a2), the CDRs differ by no more than 5, 4, 3, 2, or 1 amino acid residue(s) relative to the CDRs in the heavy chain amino acid sequence of TRL053 (SEQ ID NO: 94).

In addition or alternatively to (a1) and/or (a2), the present therapeutic formulation comprises one or more anti-influenza monoclonal antibodies comprising a heavy and a light chain, wherein the heavy chain comprises: (b1) the heavy chain amino acid sequence of TRL579 (SEQ ID NO: 96) and/or the one or more anti-influenza monoclonal antibodies comprises an antibody that binds to the same epitope as the antibody of (b1) comprising a heavy and a light chain, wherein the heavy chain comprises: (b2) a polypeptide having at least 80%, such as at least 85%, such as at least 90%, such as at least 95%, such as at least 98%, or such as at least 99% sequence identity with the heavy chain amino acid sequence of TRL579 (SEQ ID NO: 96). In certain embodiments of (b2), any amino acid substitutions, additions, or deletions in the heavy chain amino acid sequence of TRL579 (SEQ ID NO: 96) are located in the framework regions. In certain embodiments of (b2), the CDRs differ by no more than 5, 4, 3, 2, or 1 amino acid residue(s) relative to the CDRs in the heavy chain amino acid sequence of TRL579 (SEQ ID NO: 96).

In addition or alternatively to (a1), (a2), (b1) or (b2), the present therapeutic formulation comprises one or more anti-influenza monoclonal antibodies comprising a heavy and a light chain, wherein the heavy chain comprises: (c1) a heavy chain amino acid sequence selected from: (i) SEQ ID NO: 98 (TRL809); (ii) SEQ ID NO: 100 (TRL812); (iii) SEQ ID NO: 102 (TRL813); (iv) SEQ ID NO: 104 (TRL832); (v) SEQ ID NO: 106 (TRL841); (vi) SEQ ID NO: 108 (TRL842); (vii) SEQ ID NO: 110 (TRL845); (viii) SEQ ID NO: 112 (TRL846); (ix) SEQ ID NO: 114 (TRL847); (x) SEQ ID NO: 116 (TRL848); (xi) SEQ ID NO: 118 (TRL849); (xii) SEQ ID NO: 120 (TRL854); and (xiii) SEQ ID NO: 122 (TRL856) and/or wherein the one or more anti-influenza monoclonal antibodies comprises an antibody that binds to the same epitope as the antibody of (c1) comprising a heavy and a light chain, wherein the heavy chain comprises: (c2) a polypeptide having at least 80%, such as at least 85%, such as at least 90%, such as at least 95%, such as at least 98%, or such as at least 99% sequence identity with the heavy chain amino acid sequence selected from: (i) SEQ ID NO: 98 (TRL809); (ii) SEQ ID NO: 100 (TRL812); (iii) SEQ ID NO: 102 (TRL813); (iv) SEQ ID NO: 104 (TRL832); (v) SEQ ID NO: 106 (TRL841); (vi) SEQ ID NO: 108 (TRL842); (vii) SEQ ID NO: 110 (TRL845); (viii) SEQ ID NO: 112 (TRL846); (ix) SEQ ID NO: 114 (TRL847); (x) SEQ ID NO: 116 (TRL848); (xi) SEQ ID NO: 118 (TRL849); (xii) SEQ ID NO: 120 (TRL854); and (xiii) SEQ ID NO: 122 (TRL856). In certain embodiments of (c2), any amino acid substitutions, additions, or deletions in the heavy chain amino acid sequence (i) SEQ ID NO: 98 (TRL809); (ii) SEQ ID NO: 100 (TRL812); (iii) SEQ ID NO: 102 (TRL813); (iv) SEQ ID NO: 104 (TRL832); (v) SEQ ID NO: 106 (TRL841); (vi) SEQ ID NO: 108 (TRL842); (vii) SEQ ID NO: 110 (TRL845); (viii) SEQ ID NO: 112 (TRL846); (ix) SEQ ID NO: 114 (TRL847); (x) SEQ ID NO: 116 (TRL848); (xi) SEQ ID NO: 118 (TRL849); (xii) SEQ ID NO: 120 (TRL854); and (xiii) SEQ ID NO: 122 (TRL856) are located in the framework regions. In certain embodiments of (c2), the CDRs differ by no more than 5, 4, 3, 2, or 1 amino acid residue(s) relative to the CDRs in the heavy chain amino acid sequence of (i) SEQ ID NO: 98 (TRL809); (ii) SEQ ID NO: 100 (TRL812); (iii) SEQ ID NO: 102 (TRL813); (iv) SEQ ID NO: 104 (TRL832); (v) SEQ ID NO: 106 (TRL841); (vi) SEQ ID NO: 108 (TRL842); (vii) SEQ ID NO: 110 (TRL845); (viii) SEQ ID NO: 112 (TRL846); (ix) SEQ ID NO: 114 (TRL847); (x) SEQ ID NO: 116 (TRL848); (xi) SEQ ID NO: 118 (TRL849); (xii) SEQ ID NO: 120 (TRL854); and (xiii) SEQ ID NO: 122 (TRL856).

Typically, the present formulation includes at least three anti-influenza monoclonal antibodies comprising a heavy and a light chain, wherein

(i) the heavy chain of the first antibody comprises: the heavy chain amino acid sequence of TRL053 (SEQ ID NO: 94);

(ii) the heavy chain of the second antibody comprises: the heavy chain amino acid sequence of TRL579 (SEQ ID NO: 96); and

(iii) the heavy chain of the third antibody comprises the heavy chain amino acid sequence of TRL849 (SEQ ID NO: 118).

In some embodiments, the present therapeutic formulation comprises one or more anti-influenza monoclonal antibodies comprising a heavy and a light chain, wherein the heavy and the light chain comprise: (a1) a heavy chain amino acid sequence of TRL053 (SEQ ID NO: 94) and a light chain amino acid sequence of TRL053 (SEQ ID NO: 95); and/or wherein the one or more anti-influenza monoclonal antibodies comprises an antibody that binds to the same epitope as the antibody of (a1) comprising a heavy and a light chain, wherein the heavy chain and the light chain comprise: (a2) a polypeptide having at least 80%, such as at least 85%, such as at least 90%, such as at least 95%, such as at least 98%, or such as at least 99% sequence identity with the heavy chain amino acid sequence of TRL053 (SEQ ID NO: 94) and the light chain amino acid sequence of TRL053 (SEQ ID NO: 95). In certain embodiments of (a2), any amino acid substitutions, additions, or deletions in the heavy chain and light chain amino acid sequences of TRL053 (SEQ ID NO: 94 and SEQ ID NO: 95, respectively) are located in the framework regions. In certain embodiments of (a2), the CDRs differ by no more than 5, 4, 3, 2, or 1 amino acid residue(s) relative to the CDRs in the heavy chain and light chain amino acid sequences of TRL053 (SEQ ID NO: 94 and SEQ ID NO: 95, respectively).

In addition or alternatively to (a1) and/or (a2), the present therapeutic formulation comprises one or more anti-influenza monoclonal antibodies comprising a heavy and a light chain, wherein the heavy and the light chain comprise: (b1) a heavy chain amino acid sequence of TRL579 (SEQ ID NO: 96) and a light chain amino acid sequence of TRL579 (SEQ ID NO: 97) and/or wherein the one or more anti-influenza monoclonal antibodies comprises an antibody that binds to the same epitope as the antibody of (b1) comprising a heavy and a light chain, wherein the heavy and light chain comprise: (b2) a polypeptide having at least 80%, such as at least 85%, such as at least 90%, such as at least 95%, such as at least 98%, or such as at least 99% sequence identity with the heavy chain amino acid sequence of TRL579 (SEQ ID NO: 96) and the light chain amino acid sequence of TRL579 (SEQ ID NO: 97). In certain embodiments of (b2), any amino acid substitutions, additions, or deletions in the heavy chain and light chain amino acid sequences of TRL579 (SEQ ID NO: 96 and SEQ ID NO: 97, respectively) are located in the framework regions. In certain embodiments of (b2), the CDRs differ by no more than 5, 4, 3, 2, or 1 amino acid residue(s) relative to the CDRs in the heavy chain and light chain amino acid sequences of TRL579 (SEQ ID NO: 96 and SEQ ID NO: 97, respectively).

In addition or alternatively to (a1), (a2), (b1) or (b2), the present therapeutic formulation comprises one or more anti-influenza monoclonal antibodies comprising a heavy and light chain, wherein the heavy and the light chain comprise: (c1) (i) SEQ ID NOS: 98, 99 respectively (TRL809); (ii) SEQ ID NOS: 100, 101 respectively (TRL812); (iii) SEQ ID NOS:102, 103 respectively (TRL813); (iv) SEQ ID NOS: 104, 105 respectively (TRL832); (v) SEQ ID NOS: 106, 107 respectively (TRL841); (vi) SEQ ID NOS: 108, 109 respectively (TRL842); (vii) SEQ ID NOS: 110, 111 respectively (TRL845); (viii) SEQ ID NOS: 112, 113 respectively (TRL846); (ix) SEQ ID NOS: 114, 115 respectively (TRL847); (x) SEQ ID NOS: 116, 117 respectively (TRL848); (xi) SEQ ID NOS: 118, 119 respectively (TRL849); (xii) SEQ ID NOS: 120; 121 respectively (TRL854); and (xiii) SEQ ID NOS: 122, 123 respectively (TRL856) and/or wherein the one or more anti-influenza monoclonal antibodies comprises an antibody that binds to the same epitope as the antibody of (c1) comprising a heavy and a light chain, wherein the heavy and the light chain comprise: (c2) a polypeptide having at least 80%, such as at least 85%, such as at least 90%, such as at least 95%, such as at least 98%, or such as at least 99% sequence identity with the heavy and the light chain sequences, selected from: (i) SEQ ID NOS: 98, 99 respectively (TRL809); (ii) SEQ ID NOS: 100, 101 respectively (TRL812); (iii) SEQ ID NOS: 102, 103 respectively (TRL813); (iv) SEQ ID NOS: 104, 105 respectively (TRL832); (v) SEQ ID NOS: 106, 107 respectively (TRL841); (vi) SEQ ID NOS: 108, 109 respectively (TRL842); (vii) SEQ ID NOS: 110, 111 respectively (TRL845); (viii) SEQ ID NOS: 112, 113 respectively (TRL846); (ix) SEQ ID NOS: 114, 115 respectively (TRL847); (x) SEQ ID NOS: 116, 117 respectively (TRL848); (xi) SEQ ID NOS: 118, 119 respectively (TRL849); (xii) SEQ ID NOS: 120, 121 respectively (TRL854); and (xiii) SEQ ID NOS: 122, 123 respectively (TRL856). In certain embodiments of (c2), any amino acid substitutions, additions, or deletions in the heavy chain and light chain amino acid sequences (i) SEQ ID NOS: 98, 99 respectively (TRL809); (ii) SEQ ID NOS: 100, 101 respectively (TRL812); (iii) SEQ ID NOS: 102, 103 respectively (TRL813); (iv) SEQ ID NOS: 104, 105 respectively (TRL832); (v) SEQ ID NOS: 106, 107 respectively (TRL841); (vi) SEQ ID NOS: 108, 109 respectively (TRL842); (vii) SEQ ID NOS: 110, 111 respectively (TRL845); (viii) SEQ ID NOS: 112, 113 respectively (TRL846); (ix) SEQ ID NOS: 114, 115 respectively (TRL847); (x) SEQ ID NOS: 116, 117 respectively (TRL848); (xi) SEQ ID NOS: 118, 119 respectively (TRL849); (xii) SEQ ID NOS: 120, 121 respectively (TRL854); and (xiii) SEQ ID NOS: 122, 123 respectively (TRL856) are located in the framework regions. In certain embodiments of (c2), the CDRs differ by no more than 5, 4, 3, 2, or 1 amino acid residue(s) relative to the CDRs in the heavy chain and light chain amino acid sequences of (i) SEQ ID NOS: 98, 99 respectively (TRL809); (ii) SEQ ID NOS: 100, 101 respectively (TRL812); (iii) SEQ ID NOS: 102, 103 respectively (TRL813); (iv) SEQ ID NOS: 104, 105 respectively (TRL832); (v) SEQ ID NOS: 106, 107 respectively (TRL841); (vi) SEQ ID NOS: 108, 109 respectively (TRL842); (vii) SEQ ID NOS: 110, 111 respectively (TRL845); (viii) SEQ ID NOS: 112, 113 respectively (TRL846); (ix) SEQ ID NOS: 114, 115 respectively (TRL847); (x) SEQ ID NOS: 116, 117 respectively (TRL848); (xi) SEQ ID NOS: 118, 119 respectively (TRL849); (xii) SEQ ID NOS: 120, 121 respectively (TRL854); and (xiii) SEQ ID NOS: 122, 123 respectively (TRL856).

Typically, the present formulation includes at least three anti-influenza monoclonal antibodies comprising a heavy and a light chain, wherein

(i) the heavy and the light chain of the first antibody comprises, respectively: the heavy chain amino acid sequence of TRL053 (SEQ ID NO: 94) and the light chain amino acid sequence of TRL053 (SEQ ID NO: 95);

(ii) the heavy and the light chain of the second antibody comprises, respectively: the heavy chain amino acid sequence of TRL579 (SEQ ID NO: 96) and the light chain amino acid sequence of TRL579 (SEQ ID NO: 97); and

(iii) the heavy and light chain of the third antibody comprise: the heavy chain amino acid sequence of TRL849 (SEQ ID NO: 118) and the light chain amino acid sequence of TRL849 (SEQ ID NO: 119).

In some embodiments, the present antibodies exhibit the following binding affinity (KD) for influenza A virus, influenza B virus or hemagglutinin proteins derived therefrom, such as recombinant hemagglutinin proteins: between about 5×10⁻⁸ M and about 5×10⁻¹² M, such as about 5×10⁻⁹ M to about 5×10⁻¹¹ M, such as about 3×10⁻⁹ M to about 3×10⁻¹¹ M. In some embodiments, the binding affinity is about 5×10⁻¹⁰ M to about 5×10⁻¹¹ M. In some embodiments, the present antibodies exhibit a KD of less than 10 nM, less than 3 nM, or less than 1 nM for recombinant hemagglutinin protein. In some embodiments, the present antibodies exhibit KDs of between 10 nM and 0.1 pM. In some embodiments, the present antibodies exhibit KDs of between 3 nM and 1pM.

As used herein, “binding affinity” in reference to antibodies refers to the KD (the equilibrium dissociation constant between the antibody and its antigen), which can be determined by various methods known in the art. For example, the KD may be measured by determining oblique-incidence reflectivity difference (OI-RD) by use of a microarray or fluidic system, e.g., by ABCAM®. See Landry et al., 2012, Assay and Drug Dev. Technol. 10: 250-259, which is herein incorporated by reference in its entirety, BIACORE® (i.e., surface plasmon resonance) or competitive binding assays.

In some embodiments, the antibodies of the present formulation lack the Fc and/or have reduced effector function. WO 2015/120097, which is herein incorporated by reference in its entirety, demonstrates that Fc function and Fc portions of neutralizing antibodies, are not required for enhanced efficacy after intranasal and/or inhalation administration. Thus, antibody fragments, such as Fab fragments, or antibodies lacking Fc or lacking effector function, are effective intranasally or upon inhalation administration. In some embodiments, antibodies lacking effector function, are not effective when administered intravenously or by intraperitoneal injection. In some embodiments, the antibodies of the present formulation may be selected from Fab, Fab′, and F(ab′)2.

The present antibodies may be derived from a recombinant protein, may be recombinantly expressed or may be derived or generated by other means or methods, including means or methods to provide neutralizing antibody within the respiratory tract, including by way of genetic material or DNA or DNA vector expression, such as by delivering DNA or RNA encoding neutralizing antibody or fragment(s) thereof.

In some embodiments, the antibodies are bispecific antibodies. Multiple technologies now exist for making a single antibody-like molecule that incorporates antigen specificity domains from two separate antibodies (bispecific antibody). Thus, a single antibody with very broad strain reactivity can be constructed using the Fab domains of individual antibodies with broad reactivity to Group 1 and Group 2 respectively, or of one of these groups in combination with binding influenza B. Suitable technologies have been described by Macrogenics (Rockville, Md.), Micromet (Bethesda, Md.) and Merrimac (Cambridge, Mass.). (See, e.g., Orcutt, K. D., et al., “A modular IgG-scFv bispecific antibody topology,” Protein Eng Des Sel. (2010) 23:221-228; Fitzgerald, J., et al., “Rational engineering of antibody therapeutics targeting multiple oncogene pathways,” MAbs. (201 1) 1:3(3); and Baeuerle, P. A., et al., “Bispecific T-cell engaging antibodies for cancer therapy,” Cancer Res. (2009) 69:4941-4944), which are each herein incorporated by reference in its entirety. Particularly useful bispecific antibodies are those that bind to multiple types of hemagglutinin protein. Particularly useful combinations are those that combine the binding specificity of TRL053/mAb53 (H1, H5 and H9) with TRL579/mAb579 (H3 and H7).

In some embodiments, the therapeutic proteins of the present formulation comprise a combination of two, three, four, five, six, seven, eight, nine, ten, or more antibodies thereof in any ratio. In some embodiments, the therapeutic proteins of the present formulation comprise from 2-10 or 3-5 antibodies on a per weight basis of approximately 10-80 wt %; 20-50 wt %; 25-40 wt %, of each antibody per total antibody weight in the composition. In a specific embodiment, the therapeutic proteins of the present formulation comprise a substantially equal dose or ratio of a first, second and third antibody at approximately 33 wt % ±3 wt % of each of first, second and third antibodies per total wt of antibody in the formulation.

In some embodiments, the therapeutic proteins of the present formulation comprise from 2-10 antibodies in a single dose, wherein a therapeutically effective amount of each antibody in the combination may be of less than 10 mg/kg body weight, of less than 5 mg/kg body weight, of less than 2 mg/kg body weight, of 1 mg/kg body weight or less. The single dose amount of each antibody in the combination may be of less than 1 mg/kg body weight, of less than 0.5 mg/kg, of less than 0.1 mg/kg, of less than 0.05 mg/kg. Multiple doses of the antibody combination may be administered. Each combination dose may be the same or the doses may differ, such as an initial higher dose, followed by lower doses, or an initial lower dose, followed by higher doses. The single dose or doses or any dose may be of less than 1 mg/kg body weight, of less than 0.5mg/kg, of less than 0.1 mg/kg, of less than 0.05 mg/kg. The initial dose may be greater than 1 mg/kg and further or subsequent doses may be lower or may be less than 1 mg/kg.

In some embodiments, a dose of each antibody in the formulation that is intended for respiratory tract administration, particularly intranasal administration or inhalation administration, is in an amount less than 1 mg/kg on the basis of the body weight of a mammal. In some embodiments, formulations are provided comprising antibody amounting to administration of less than 10 mg/kg, less than 5 mg/kg, or less than 1 mg/kg on the basis of the body weight of a human. Formulations of the present disclosure may particularly comprise a dose of antibody that is intended for administration, particularly intranasally or via inhalation, in an amount less than 1 mg/kg, less than 0.5 mg/kg, less than 0.1 mg/kg, less than 0.05 mg/kg, less than 0.01 mg/kg, less than 0.005 mg/kg, less than 0.0025 mg/kg, less than 0.001 mg/kg on the basis of the body weight of a mammal, including a clinically relevant mammal, such as a mouse, dog, horse, cat or a human. In some embodiments, a therapeutically effective dose is selected from about 100 mg/kg, 50 mg/kg, 10 mg/kg, 3 mg/kg, 1 mg/kg, or 1 mg/kg. In some aspects, an effective prophylactic dose or post-exposure prophylactic dose is selected from about 1 mg/kg/ 0.1 mg/kg or about 0.01 mg/kg.

One of skill in the art can determine, including on the basis of efficacy in animal models and in consideration of clinical and physiological response, viral load and viral transmission rates, the appropriate and efficacious dose in a mammal, including a human. Thus, the disclosure and dosing parameters are not limited by the disclosure.

### Aerosolization

In some embodiments, the present formulation produces liquid particles upon aerosolization. Aerosolization is the process of forming an aerosol. As used herein “an aerosol” comprises a continuous gas phase and, dispersed therein, a discontinuous or dispersed phase of liquid particles or droplets. The liquid particles or droplets of the dispersed phase comprise the therapeutic protein of the present formulation in a liquid environment. The liquid environment is mainly an aqueous phase with the further excipients as described herein. The continuous gas phase of the aerosol may be selected from any gas or mixture of gases. Typically, the gas or mixture is pharmaceutically acceptable. For example, the gas may be air or compressed air. Alternatively, other gases and gas mixtures, such as air enriched with oxygen, carbon dioxide, or mixtures of nitrogen and oxygen may be used. Typically, the gas is air or compressed air.

In some embodiments, a membrane (or mesh) nebulizer is used to generate the aerosol of the disclosure. A “nebulizer” as defined herein is a device which is capable of aerosolizing a liquid material into a dispersed liquid phase.

In some embodiments, the present liquid formulation is nebulized by vibrating the present formulation. Such an oscillating fluid membrane nebulizer comprises a reservoir in which a liquid to be nebulized is filled. When operating the nebulizer, the liquid is fed to a membrane via a liquid feed system that is made to oscillate (i.e. vibrate, e.g. by means of a piezoelectric element). In some embodiments, such liquid feed system includes vibrating a back wall of the reservoir (e.g. AEROVECTRX™ Technology, Pfeifer Technology) or vibrating a liquid transporting slider (e.g. I-NEB™ device from Respironics, or U22™ device from Omron). Such nebulizers are referred to herein as “passive membrane nebulizers”.

In other embodiments, the present formulation is nebulized by vibrating the membrane (“vibrating mesh nebulizer”). Nebulizers of this type comprise a reservoir that is filled with the liquid to be nebulized. When operating the nebulizer, a liquid, e.g., the formulation of the present disclosure, is fed to a membrane that is made to oscillate, i.e. vibrate (e.g. by means of a piezoelectric element). The liquid present at one side of the vibrating membrane is transported through openings in the vibrating membrane (also referred to as “pores” or “holes”) and takes the form of an aerosol on the other side of the vibrating membrane. (e.g. EFLOW™ rapid and ERAPID™ from PARI, HL100 from Health and Life as well as AEROGEN® Go and AEROGEN® Solo Ultrasonic Nebulizer (Aerogen, Inc., Ireland). Such nebulizers are referred to herein as “active membrane nebulizers.”

Different membrane types are available for the nebulization of liquids with a membrane nebulizer. These membranes are characterized by different pore sizes, which generate aerosols with different particle sizes. Depending on the desired aerosol characteristics, different membrane types (i.e. different modified membrane nebulizers or aerosol generators) can be used.

Two values can be determined experimentally and used to describe the particle size of the generated aerosol: the mass median diameter (MMD) and the mass median aerodynamic diameter (MMAD). The difference between the two values is that the MMAD is normalized to the density of water. The MMAD may be measured by an impactor, for example the Anderson Cascade Impactor (ACI) or the Next Generation Impactor (NGI). Alternatively, laser diffraction methods may be used, for example the MALVERN MASTERSIZER X™, to measure the MMD.

Another parameter describing the dispersed phase of the aerosol is the particle size distribution of the aerosolized liquid particles. The geometric standard deviation (GSD) is an often used measure for the broadness of the particle or droplet size distribution of generated aerosol particles or droplets.

For aerosol delivery into the respiratory tract, the particles include an MMAD ranging from 1 μm to 11 μm, such as 1.5 μm to 5 μm, such as 2.3 μm to 4.5 μm. In some embodiments, the MMAD is below 10.0 μm, such as below 5.0 μm, such as below 3.3 μm or such as below 2.3 μm. In some embodiments, the size distribution has a GSD ranging from 1 to 3, such as from 1.5 to 2.5, such as from 1.8 to 2.3. In some embodiments, the size distribution has a GSD of less than 2.3, typically less than 2.0, more typically less than 1.8 or even more typically less than 1.6. Such particle size distribution is particularly useful to achieve a high local therapeutic protein concentration in the respiratory tract of humans, relative to the amount of therapeutic protein which is aerosolized.

The selection of a precise particle size of the foregoing particle size ranges should take the target region or tissue for deposition of the aerosol into account. For example, the optimal droplet diameter will differ depending on whether nasal or oral inhalation is intended, and whether upper (e.g., nostrils, nasal cavity, mouth, throat (pharynx), and voice box (larynx) and/or lower respiratory tract delivery (e.g. trachea, lungs, bronchi, alveoli) is focused upon. Additionally, the age-dependent anatomic geometry (e.g. the nose, mouth or respiratory airway geometry) as well as the respiratory disease and condition of the patients and their breathing pattern belong to the factors determining the optimal particle size (e.g. MMAD) for therapeutic protein delivery to the lower or upper respiratory tract.

In some embodiments, the aerosol is for upper respiratory tract delivery, in particular, the nose, nasal and/or sinonasal mucosa, osteomeatal complex, and paranasal cavities. In these embodiments, the MMAD ranges from about 1 μm to about 10 μm, such as about 3 μm to about 10 μm, such as about 3 μm to about 5 μm. In some embodiments the MMAD is below about 10 μm, such as below about 5.0 μm, or such as below about 4.5 μm, or such as below about 4.0 μm, or such as below about 3.3 or such as below about 3.0 μm is particularly suitable.

In some embodiments, the formulation is for lower respiratory tract delivery, in particular, deep into the lungs. Generally, small airways, which are defined by an internal diameter typically lower than 2 mm, represent almost 99% of the lung volume and therefore play a role in lung function. Alveoli are sites in the deep lungs where oxygen and carbon dioxide are exchanged with the blood. Inflammation in the alveoli induced by some viruses or bacteria leads to fluid secretion on site and directly affects oxygen uptake by the lungs. Therapeutic targeting of deep pulmonary airways with aerosols typically includes particles having an MMAD ranging from about 1 μm to about 5 μm, such as about 2 μm to about 4 μm, such as about 3 μm to about 5 μm. In some embodiments, the MMAD is below 5.0 μm, typically below 4.5 μm, such as 4.0 μm, more typically below 3.3 μm and even more typically below 3.0 μm.

In some embodiments, the aerosol is to be deposited into the lungs of children and/or infants. In these embodiments, smaller droplet sizes (MMADs) are used, ranging from e.g., about 1.0 to about 3.3 μm, more typically below 2.0 μm.

In aerosol therapy, the fraction of particles smaller than a certain size, e.g., an MMAD smaller than 5 μm (representing the fraction typically respirable by an adult), or 3.3 μm (representing the fraction typically respirable by a child or which is typically deposited in the deeper lungs of an adult) may be evaluated. Also, the fraction of particles smaller than 2 μm is often evaluated as it represents the fraction of the aerosol that could optimally reach terminal bronchioles and alveoli of adults and children and can penetrate the lungs of infants and babies.

In the some embodiments, the fraction of droplets having a particle size smaller than 5 μm is typically greater than 40%, is typically greater than 65%, more typically greater than 70% and even more typically greater than 80%. The fraction of droplets having a particle size smaller than 3.3 μm is typically greater than 25%, more typically greater than 30%, even more typically greater than 35% and still more typically greater than 40%. The fraction of droplets having a particle size smaller than 2 μm is typically greater than 4%, more typically greater than 6% and even more typically greater than 8%.

A typical membrane nebulizer for targeting the upper respiratory tract is a nebulizer which generates the aerosol via a perforated vibrating membrane principle, such as a modified investigational membrane nebulizer using the EFLOW™ technology, but which is also capable of emitting a pulsating air flow so that the generated aerosol cloud pulsates (i.e. undergoes fluctuations in pressure) at the desired location or during transporting the aerosol cloud to the desired location (e.g. sinonasal or paranasal sinuses). This type of nebulizer has a nose piece for directing the flow transporting the aerosol cloud into the nose. Aerosols delivered by such a modified electronic nebulizer may reach sinonasal or paranasal cavities much better than when the aerosol is delivered in a continuous (non-pulsating) mode. The pulsating pressure waves may achieve a more intensive ventilation of the sinuses so that a concomitantly applied aerosol is better distributed and deposited in these cavities. More particularly, a typical nebulizer for targeting the upper respiratory tract of a subject is a nebulizer adapted for generating an aerosol at an effective flow rate of less than about 5 liters minute and for simultaneously operating means for effecting a pressure pulsation of the aerosol at a frequency in the range from about 10 to about 90 Hz, wherein the effective flow rate is the flow rate of the aerosol as it enters the respiratory system of the subject. Examples of such electronic nebulization devices are disclosed in WO 2009/027095, which is herein incorporated by reference in its entirety.

In a typical embodiment of the disclosure, the nebulizer for targeting the upper respiratory tract is a nebulizer which uses a transportation flow that can be interrupted when the aerosol cloud reaches the desired location and then starts the pulsation of the aerosol cloud, e.g. in an alternating mode, such as described in WO 2010/09719 and WO 2011/134940, which are each herein incorporated by reference in its entirety. For aerosol delivery to the nose, e.g. the SINUS™ device (jet nebulizer) from PARI and also a membrane nebulizer (prototypes of VIBRENT™ technology) may be used. The suitability of the generated aerosol for application to the upper airways can be evaluated in nasal inhalation models such as the human nasal cast model described in WO 2009/027095, which is herein incorporated by reference in its entirety.

If the method is intended for targeting the lower respiratory tract such as the bronchi or the deep lungs, a piezoelectric perforated membrane-type nebulizer is typically selected for generating the aerosol. Examples of suitable nebulizers include the passive membrane nebulizer, such as 1-NEB™, U22™, U 1™, MICRO AIR™, the ultrasonic nebulizer, for example MULTISONIC™, and/or an active membrane nebulizer, such as HL1 00™, RESPIMATE™, EFLOW™ Technology nebulizers, AEROGEN® Solo Ultrasonic Nebulizer, AERONEB PRO™, AEROGEN® GO, and AEROGEN® DOSE device families as well as the Pfeifer, Chrysalis (Philip Morris) or AEROVECTRX™ devices or the EFLOW™ nebulizer (electronic vibrating membrane nebulizer available from PARI, Germany). Alternatively a passive membrane nebulizer may be used, for example U22™ or U 1™ from Omron or a nebulizer based on the Telemaq.fr technique or the Ing. Erich Pfeiffer GmbH technique.

Whether adapted for pulmonary or sinonasal delivery, the nebulizer should typically be selected or adapted to be capable of aerosolizing a unit dose at a typical output rate. A unit dose is defined herein as a volume of the present aqueous therapeutic composition comprising the therapeutically effective amount of active compound, i.e. the therapeutic protein as described herein, designated to be administered during a single administration. In some embodiments, the nebulizer can deliver such a unit dose at a rate of at least 0.1 mL/minute or at a rate of at least 50 mg/minute.

The volume of the composition that is nebulized is typically low, which helps to reduce nebulization times. The volume, also referred to as the volume of a dose, or a dose unit volume, or a unit dose volume, should be understood as the volume, which is intended for being used for one single administration or nebulizer therapy session. Specifically, the volume may be in the range from 0.3 mL to 9.0 mL, typically 0.5 mL to 6 mL, or more typically 1.0 mL to about 4.5 mL, or even more typically about 3 mL. Typically, a nebulizer, as described herein, generates an aerosol where a major fraction of the loaded dose of liquid aqueous formulation is delivered as aerosol, i.e. to have a high output. More specifically, the nebulizer generates an aerosol which contains at least 50% of the dose of therapeutic protein in the formulation, or, in other words, which emits at least 50% of the liquid aqueous formulation filled in the reservoir.

### Methods

In some embodiments, the present disclosure provides a method of generating an aerosol comprising the step of nebulizing the present therapeutic formulation, as described herein, using a nebulizer, as also herein described, to obtain an aerosol. Typically, the nebulizer is a vibrating mesh nebulizer as described herein.

In some embodiments, the present disclosure provides a method for the therapeutic and/or prophylactic treatment of influenza, such as influenza A and/or influenza B, as herein described, which method comprises administering a therapeutically effective amount of the present therapeutic formulation, as also herein described, to a subject in need thereof. In some embodiments, the present therapeutic formulation is administered intranasally. In other embodiments, the therapeutic protein formulation of the present disclosure is administered by inhalation as described herein. Typically, the administration comprises inhaling an aerosol generated by a nebulizer, as also described herein, typically a vibrating membrane nebulizer. In some embodiments, the nebulizer generates an aerosol targeting the upper respiratory tract of a subject as herein described. More typically, the nebulizer generates an aerosol targeting the lower respiratory tract of a subject as described herein.

### EXAMPLES

### Example 1. Aerosol Characteristics

Three different formulations (formulations 1, 2 and 3) having the components shown in Table 1, below, each at a pH of 6.5, were prepared and assessed for their ability to generate particles after aerosolization and for retention of activity. As indicated in Table 1, all of the formulations contained three different antibodies (a mixture of TRL053, TRL579 and TRL849 (50 mg/mL)), 20 mM histidine-chloride buffer and 115 mM NaCL. Formulations 2 and 3, unlike formulation 1, however, additionally contained polysorbate-20 in an amount of 0.02% (v/v) or 0.05% (v/v), respectively.

TABLE 1
Formulations with different amounts of polysorbate-20

| | Formulation 1 | Formulation 2 | | Formulation 3 | |
|---|---|---|---|---|---|
| | (amount of | (amount of | | (amount of | |
| Component | component) | component) | | component) | |
| Antibody mixture¹: | 50 mg/mL | 50 mg/mL | | 50 mg/mL | |
| | total antibody | total antibody | | total antibody | |
| Histidine-chloride | 20 mM | 20 | mM | 20 | mM |
| buffer | | | | | |
| NaCl | 115 mM | 115 | mM | 115 | mM |
| Polysorbate-20 | N/A | 0.02% | (v/v) | 0.05% | (v/v) |

¹Mixture of TRL053, TRL579 and TRL849)

Samples were subjected to ELISA to determine hemagglutinin-binding ability for each of the antibodies (TRL053, TRL579 and TRL849) in the formulations. Aerosolization was then performed using a nebulizer (AEROGEN® Solo Ultrasonic Nebulizer, Aerogen, Inc., Ireland). Two samples from each formulation were again subjected to ELISA to determine the hemagglutinin-binding ability of each of the antibodies after nebulization.

After aerosolization, the aerosol particles were collected at a flow rate of 15 liters/minute using a Next Generation Impactor (Copley Scientific, Inc., United Kingdom). Two samples from each formulation were then analyzed by gravimetric and ultraviolet assays.

For gravimetric assays, the weight of the particles on each impactor plate was determined by calculating the weight difference of each plate before and after nebulization. This assay, which provides a particle distribution based on the liquid droplet weight that accumulates on each plate, was then used in calculations for determining droplet particle size distribution.

For UV assays, material on each impactor plate was collected and antibody concentration determined by UV absorbance. The net distribution of antibody was then calculated. This assay, which provides a particle distribution based on the amount of protein that accumulates on each plate, may be compared to the distribution of particle droplet weight determined by the gravimetric assay.

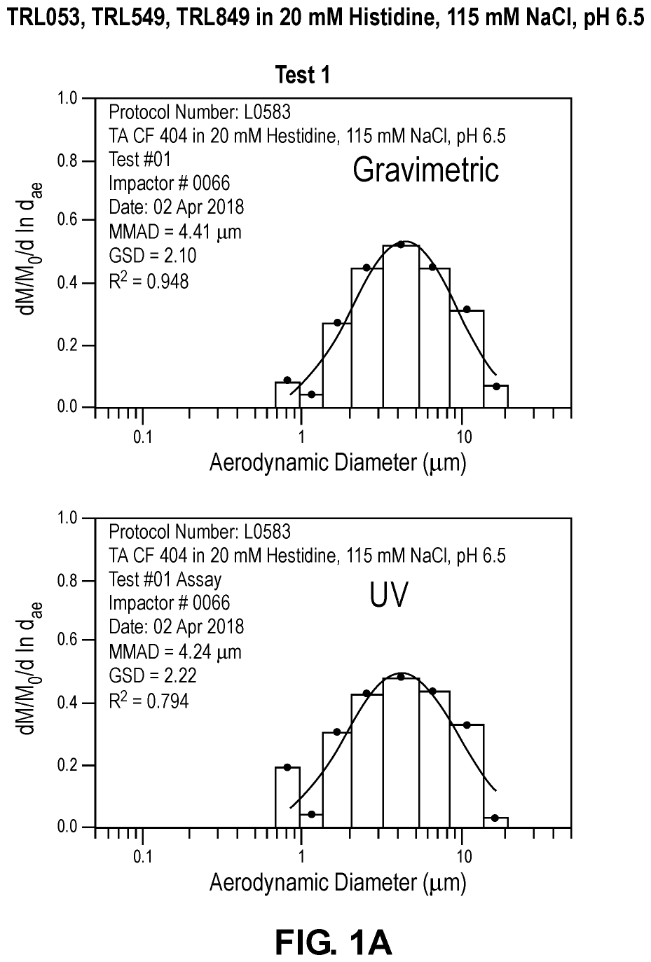

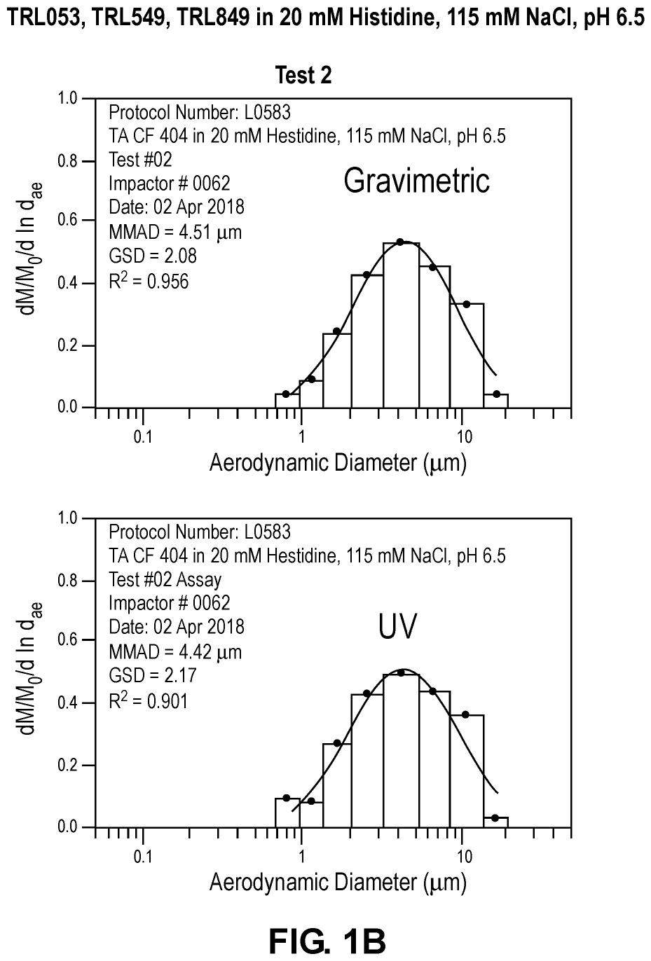

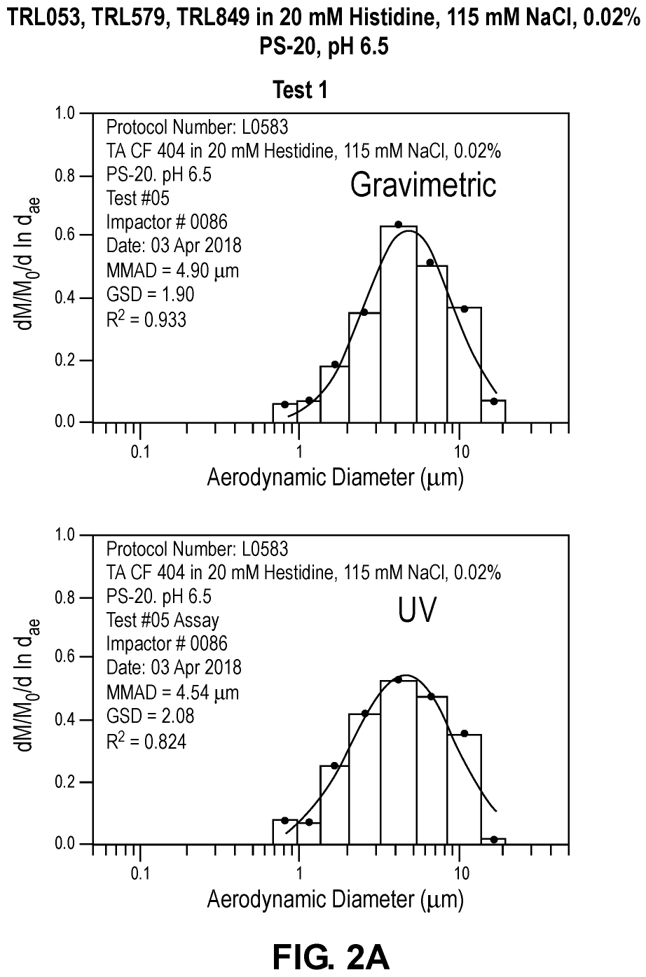

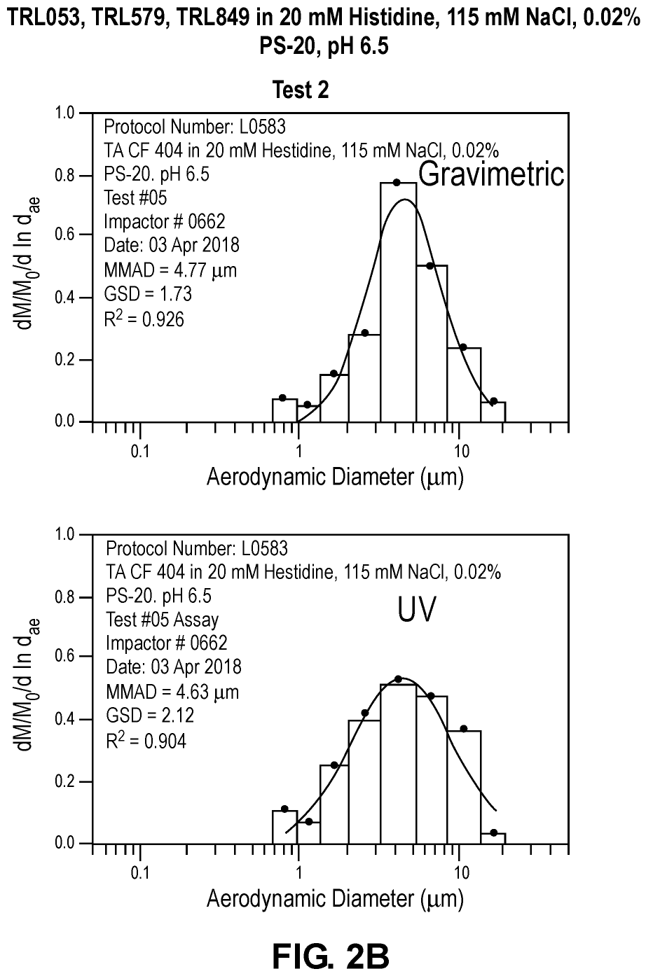

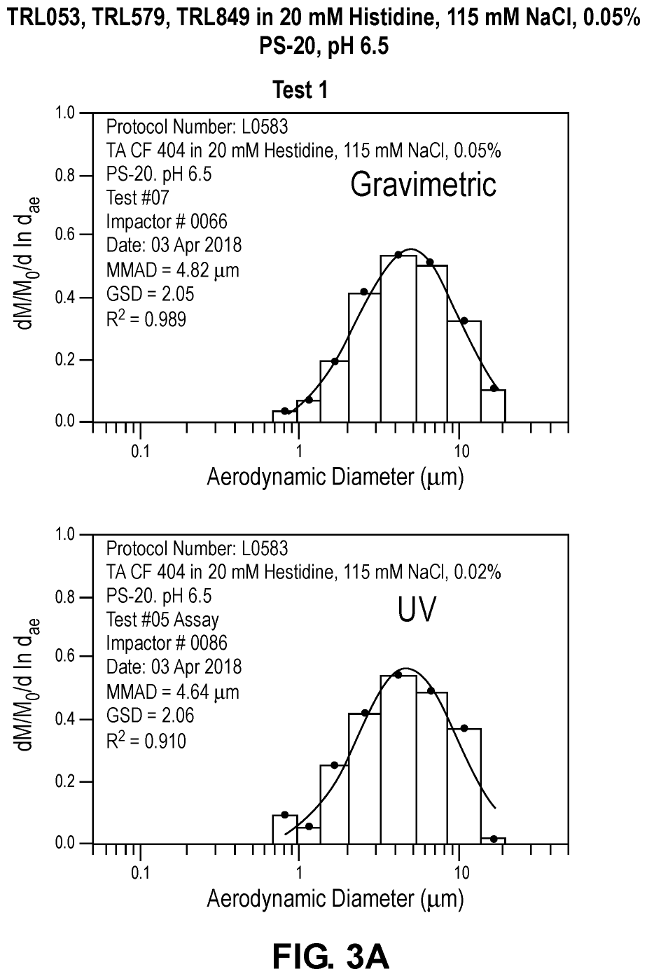

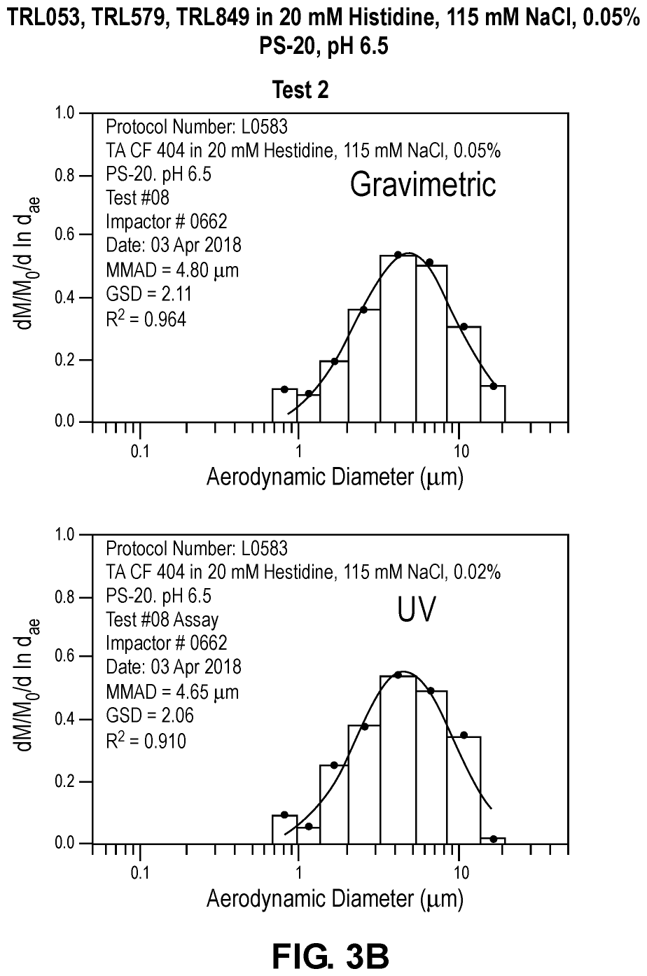

FIGS. 1-3 depict histograms of particle size distribution for formulations 1, 2 and 3, respectively. Changes in the formulation did not have a marked impact on particle size or particle size distribution. All formulations generated particles sizes (MMAD) less than 5 μm (4.24 μm -4.90 μm), indicating that the formulations are suitable for administration into the respiratory tract, including deep lung penetration. In addition, the geometric standard deviation (GSD) values ranged from 1.73 to 2.22, further indicating a particle size distribution suitable to achieve a high local therapeutic protein concentration deep in the respiratory tract of humans.

Moreover, as indicated in Table 2, below, the nebulized samples from all formulations demonstrated the ability to bind hemagglutinin. Further, as is also evident from the table, the amount of antibody, which exhibited hemagglutinin-binding ability before nebulization was comparable within experimental limits to the amount of antibody that exhibited hemagglutinin binding after nebulization.

TABLE 2
Concentrations of antibody that bind to hemagglutinin
before and after nebulization²

| Amount of PS-20 in formulation | | | |
|---|---|---|---|
| (20 mM histidine buffer, 115 mM | | Pre-Neb³ | Post-Neb⁴ |
| NaCl and PS-20, pH 6.5) | Antibody | (mg/mL) | (mg/mL) |
| 0.02% | TRL053 | 21.00 | 18.30 |
| | | 20.80 | 18.90 |
| 0.05% | | 16.70 | 19.00 |
| | | 17.90 | 18.30 |
| 0.02% | TRL579 | 21.30 | 19.70 |
| | | 21.90 | 20.30 |
| 0.05% | | 18.70 | 20.20 |
| | | 20.10 | 20.10 |
| 0.02% | TRL849 | 21.70 | 22.80 |
| | | 21.70 | 21.80 |
| , 0.05% | | 19.20 | 24.00 |
| | | 18.20 | 17.40 |

²Determined by ELISA
³Pre-Neb = pre-nebulization
⁴Post-Neb = post-nebulization

### Example 2. Storage

ELISA

A formulation of the disclosure was also assessed for its ability to stabilize antibody and to allow for activity after storage. To assess these properties, a solution was initially prepared, which contained 20 mM histidine-chloride buffer, 115 mM NaCl and 0.02% (v/v) polysorbate 20, pH 6.5. A combination of TRL053 (18.6 mg/mL), TRL579 (18.3 mg/mL) and TRL849 (18.1 mg/mL was then added to the solution. The formulation was subsequently stored for three months at 2° C. to 8° C. or −70° C. to determine short term stability. After storage, the formulation was subjected to ELISA and the concentration of each antibody retaining binding ability was determined.

Table 3 describes the concentrations of each of the three antibodies, TRL053, TRL579, TRL849, before and after storage. As shown in Table 3, the measured concentrations increased (TRL053, TRL849) or decreased (TRL579) after storage at 2° C. to 8° C. or −70° C. in comparison to the before storage nominal concentrations. Nevertheless, the after storage measured concentrations were comparable to the before storage nominal concentrations within experimental limits. Further, the concentrations after storage, as determined by ELISA, indicate the amount of each antibody that retains antigen-binding ability. Accordingly, the present formulations may be used to stabilize antibodies during storage for at least three months without apparent loss of activity.

TABLE 3
Formulation buffer concentrations of TRL053,
TRL579 or TRL849 after three months storage

| | | Theoretical | |
|---|---|---|---|
| | | concentration | ELISA |
| | Storage | prior to storage | Concentration |
| Antibody | Temperature | (mg/mL) | (mg/mL) |
| TRL053 | 2° C.-8° C. | 18.6 | 19.2 |
| | −70° C. | | 21.5 |
| TRL579 | 2° C.-8° C. | 18.3 | 14.8 |
| | −70° C. | | 16.3 |
| TRL849 | 2° C.-8° C. | 18.1 | 19.6 |
| | −70° C. | | 21.4 |

UV

In order to assess the antibody concentration in the formulation after storage more accurately than that determined by ELISA, the concentration of total antibody was determined directly using UV absorbance. As indicated in Table 4, below, the nominal concentration of the total amount of antibody before storage (56.79 mg/mL) was comparable to the concentration of the total antibody determined using UV absorbance after three months at 2° C.-8° C. (58.30 mg/mL) and −70° C. 58.93 mg/mL). Accordingly, these data further demonstrate that there is no apparent degradation of the antibodies during the storage conditions when formulated in accordance with the present disclosure.

Table 4 further indicates that, unlike the formulation before storage, granular particles are visually observed in the formulations after storage. Nevertheless, as noted above, the ELISA data demonstrate that antibody activity is retained after storage, indicating the lack of impact of any apparent aggregation. Further, as also noted in Table 4, the pH is maintained during storage, thus further supporting that the present formulation is effective in maintaining conditions during storage sufficient to stabilize the antibodies.

TABLE 4
Concentration, Appearance and pH of Antibody combination (TRL053,
TRL579 and TRL849) in the present formulation after storage

| | | T = 3 months | |
|---|---|---|---|
| Method | T = 0 | 2-8° C. | −70° C. |
| UV | 56.79 mg/mL | 58.30 mg/mL | 58.93 mg/mL |
| Appearance | No particles | Granular particles | Granular particles |
| pH | 6.54 | 6.52 | 6.53 |

### Example 3. Safety and Efficacy

Male Sprague Dawley rats were used to conduct a single dose inhalation safety, toxicity and toxicokinetic (TK) profile study of the TRL053, TRL579 and TRL849 combination antibody formulation according to the present disclosure. Initially, the aerosol generation conditions were established. Reproducible aerosol was generated in a side-stream nebulizer using 55 mg/mL of the combination antibody formulation, resulting in an aerosol concentration of 1.2 mg//mL and a total dosage of 308 to 320 mg/mL over a six hour exposure period.

The generated atmospheres and particle size distributions were deemed acceptable for the rat inhalation studies. The oxygen concentration and temperature range of the generated atmospheres was 20.9% and 19-24° C., respectively, with a relative humidity between 42.6% and 70.4%. Further, the particle size distribution indicated that the aerosols were respirable (MMAD about 2 μm, GSD about 2 μm).

Groups of male Sprague Dawley rats were exposed to a single nose-only inhalation of a liquid aerosol comprising the antibody combination formulation or vehicle control. As shown below in Table 5, rats were exposed to the aerosolized formulations for 90 minutes (low dose), 180 minutes (mid dose) and 360 minutes (high dose and vehicle control).

TABLE 5
Single Inhalation Administration to Sprague Dawley rats

| | | | Target | Target Aerosol | | |
|---|---|---|---|---|---|---|
| | | | Total | Concentration of | Toxicology | |
| | | Exposure | Delivered | antibody | Main | TK |
| Group | Group | Duration | Dose Level | combination | Animals | Animals |
| No. | Designation | (minutes) | (mg/kg) | (mg/L)^{a} | Males | Males |
| 1 | Vehicle | 360 | 0 | 0 | 6 | 3 |
| | Control | | | | | |
| 2 | Low Dose | 90 | 81 | 1.2 | 6 | 9 |
| 3 | Mid Dose | 180 | 161 | 1.2 | 6 | 9 |
| 4 | High Dose | 360 | 322 | 1.2 | 6 | 9 |

^{a}Targeted aerosol concentrations were calculated based on an estimated body weight of 0.250 kg.

The mean achieved test atmosphere concentrations of the antibody formulation, as assessed by chemical determination, were as follows.

TABLE 6
Test Atmosphere Concentration and Estimated Achieved Dose Levels

| | | Mean Test Atmosphere | | | | |
|---|---|---|---|---|---|---|
| Group | | Concentration (mg/L) | | | | % of Target |
| No. | Group Designation | Targeted | Achieved | SD | % RSD | Achieved |
| 1 | Vehicle Control | 0.00 | BLQ | NA | NA | NA |
| 2 | Low Dose | 1.2 | 0.948^{a} | 0.1164 | 12.3 | 79 |
| 3 | Mid Dose | 1.2 | 0.989^{a} | 0.1101 | 11.1 | 82.4 |
| 4 | High Dose | 1.2 | 0.971^{a} | 0.1130 | 11.6 | 80.9 |

SD = Standard Deviation,
RSD = Relative Standard Deviation
BLQ: Below limit of quantification,
NA: Not applicable
^{a}Individual aerosol concentrations (total of 12) were: 0.774, 1.003, 0.999, 1.017, 1.072, 1.069, 1.042, 0.721, 0.942, 0.945, 1.015, and 1.054 mg/L. From the samples collected, the first 4 represented Group 2, first 6 represented Group 3, and all 12 represented Group 4.
% of Target Achieved = (Mean Achieved aerosol concentration/Targeted aerosol concentration) × 100

Particle size distributions of the aerosolized vehicle control, as assessed by gravimetric determination, for Group 1 and by chemical determination for Groups 2 to 4 were as follows.

TABLE 7
Particle Size Distribution (PSD) Measurements.

| | |
|---|---|
| Group | Particle Size |

| No. | MMAD (μm) | GSD |
|---|---|---|
| 1 | 3.9 | 2.05 |
| 1 | 2.8 | 1.86 |
| 1 | 2.2 | 2.06 |
| 2 | 1.9 | 1.90 |
| 2 | 1.4 | 2.43 |
| 3 | 2.0 | 1.81 |
| 4 | 1.8 | 1.88 |

MMAD = Mass median aerodynamic diameter.
GSD = Geometric standard deviation.

The particle size distribution measurements confirmed that the antibody combination in the aerosolized test item was respirable for the rat. The deposition within the respiratory tract was considered to be 100% as the mean Mass Median Aerodynamic Diameters (MMAD) were ≤2.0 μm with corresponding geometric standard deviations (σg)<2.5 for Groups 2, 3 and 4. Similarly, the aerosolized vehicle control for Group 1 was considered respirable with the MMAD ranging between 2.2 to 3.9 μm and GSD ranging between 1.86 and 2.06.

The particle size distribution measurements confirmed that the antibody combination in the aerosolized test item was respirable for the rat. The deposition within the respiratory tract was considered to be 100% as the mean Mass Median Aerodynamic Diameters (MMAD) were less than or equal to 2.0 μm with corresponding geometric standard deviations (σg) less than 2.5 for Groups 2, 3 and 4. Similarly, the aerosolized vehicle control for Group 1 was considered respirable with the MMAD ranging between 2.2 to 3.9 μm and GSD ranging between 1.86 and 2.06.

As shown in Table 8, below, the following delivered total and pulmonary dosages, respectively, were estimated for each of the groups: 62 mg/kg, 6.2 mg/kg (Group 2), 129 mg/kg, 12.9 mg/kg (Group 3) and 255 mg/kg, 25.5 mg/kg (Group 4). The overall estimated achieved total and pulmonary doses were 76.5%, 80.1% and 79.2% of the targeted dose levels for the low, mid and high dose groups, respectively.

TABLE 8
Estimated Overall Achieved Doses on Day 1

| | | | | Estimated | Estimated | |
|---|---|---|---|---|---|---|
| | Target | Target Total | | Achieved | Achieved Total | |
| | Pulmonary | Delivered | Mean Body | Pulmonary Dose | Delivered Dose | |
| Group | Dose Level | Dose Level | Weight* | Level** | Level | % |
| No. | (mg/kg) | (mg/kg) | (kg) | (mg/kg) | (mg/kg) | Accuracy |
| 1 | 0 | 0 | 0.296 | 0 | 0 | NA |
| 2 | 8.1 | 81 | 0.305 | 6.2 | 62 | 76.5 |
| 3 | 16.1 | 161 | 0.299 | 12.9 | 129 | 80.1 |
| 4 | 32.2 | 322 | 0.298 | 25.5 | 255 | 79.2 |

*Mean body weight collected from Day 1 was used for estimation.
**Estimated achieved lung dose levels were calculated as 10% of the respective estimated achieved inhaled dose level of the TRL053, TRL579 and TRL849 combination.

The highest concentration of TRL053, TRL579 and TRL849 in the lungs was detected at 4 hours post dose. The concentrations of TRL053, TRL579 or TRL849 in the lungs declined gradually, but were still significantly higher than Below the Limit of Quantification (BLQ: 10.3 ng/mL) at the last collection time (168 hours post dose). Further, TRL053, TRL579 and TRL849 levels in the right caudal lobe were similar to those of the right cranial lobe, confirming that the antibody formulation can be delivered throughout the lung. The concentration of TRL053, TRL579 and TRL849 increased with increasing dosage level and at a given dose level, the concentrations of TRL053, TRL579 and TRL849 were comparable.

Systemic exposure to the TRL053, TRL579 and TRL849 combination increased in a less than dose proportional manner for all groups. However, the Cmax between the mid and high doses of TRL579 increased proportionally to the dose (2-fold). At all dose levels, the exposure to TRL053, TRL579 and TRL849 was similar, ranging from 1.0 to 1.5-fold between each antibody. This is consistent with the results obtained during the bioanalytical method where a cross reactivity of TRL053 with TRL849coating antigen was demonstrated and resulted in an average of 50% over recovery for TRL849.

There were no related clinical signs or clear effects on body weight or food consumption, ophthalmoscopy, respiratory parameter measurements or clinical pathology (hematology, coagulation, clinical chemistry and urinalysis) in any treated group.

In conclusion, based on the parameters examined, the formulation and the antibodies had no adverse effect observed at the highest tested dose (255 mg/kg). Consequently, the No Observed Adverse Effect Level (NOAEL) was considered to be at the achieved total delivered dose level of 255 mg/kg of CF-404 (CF-401: Tₘₐₓ=72 hr, Cₘₐₓ=1797 ng/mL, ACU_{0-Tlast}=229198 hr*ng/mL; CF-402: Tₘₐₓ=72 hr, Cₘₐₓ=2407 ng/mL, AU_{0-Tlast}=274663 hr*ng/mL; CF-403: Tₘₐₓ=72 hr, Cₘₐₓ=2610 ng/mL, AU_{0-Tlast}=326062 hr*ng/mL) for male rats when dosed for a single exposure by nose-only inhalation for 360 minutes.

1. An aqueous therapeutic protein formulation comprising:
(i) one or more therapeutic proteins, wherein the one or more therapeutic proteins comprise one or more anti-influenza antibodies in an amount ranging from 30 to 150 mg/mL;
(ii) histidine buffer,
(iii) NaCl, and
(iv) an aqueous carrier,
wherein a pH of the aqueous therapeutic formulation ranges from 5.5-8.0, and wherein the formulation is formulated for respiratory tract delivery and produces particles comprising the one or more therapeutic proteins upon aerosolization.

2. The aqueous therapeutic protein formulation of claim 1, wherein the histidine buffer comprises histidine chloride.

3. The aqueous therapeutic protein formulation of claim 1, wherein the amount of the therapeutic protein ranges from 40 mg/mL to about 100 mg/mL.

4. The aqueous therapeutic protein formulation of claim 1, wherein the amount of therapeutic protein is about 50 mg/mL.

5. The aqueous therapeutic protein formulation of claim 1, wherein the NaCl is at a concentration of about 115 mM.

6. The aqueous therapeutic protein formulation of claim 1, wherein the formulation further comprises a non-ionic surfactant.

7. The aqueous therapeutic protein formulation of claim 6, wherein the non-ionic surfactant comprises a polysorbate.

8. The aqueous therapeutic protein formulation of claim 7, wherein the polysorbate comprises polysorbate 20 in an amount of about 0.02%.

9. The aqueous therapeutic protein formulation of claim 1, wherein the pH of the aqueous formulation ranges from 6.0-6.5.

10. The aqueous therapeutic protein formulation of claim 1, wherein the pH of the formulation is about 6.0.

11. The aqueous therapeutic protein formulation of claim 1, wherein histidine is present in the formulation at a concentration of about 20 mM.

12. The aqueous therapeutic protein formulation of claim 1,
wherein the one or more anti-influenza antibodies comprises: a heavy and a light chain comprising, respectively (a1) the TRL053 heavy chain CDR sequences HCDR1/HCDR2/HCDR3 (SEQ ID NOS: 1, 2, 3, respectively) and the TRL053 light chain CDR sequences LCDR1/LCDR2/LCDR3 (SEQ ID NOS: 4, 5, 6, respectively); and/or
wherein the one or more anti-influenza antibodies binds to the same epitope as the antibody of (a1) and comprises (a2) a heavy and a light chain, wherein the heavy chain comprises: (a2) a polypeptide having at least 80% sequence identity with the TRL053 heavy chain CDR sequences HCDR1/HCDR2/HCDR3 (SEQ ID NOS: 1, 2, 3, respectively) and the TRL053 light chain CDR sequences LCDR1/LCDR2/LCDR3 (SEQ ID NOS: 4, 5, 6, respectively); and/or
wherein the one or more anti-influenza antibodies comprises: a heavy and a light chain comprising (b1) the TRL579 heavy chain CDR sequences HCDR1/HCDR2/HCDR3 (SEQ ID NOS: 7, 8, 9, respectively) and the TRL579 light chain CDR sequences LCDR1/LCDR2/LCDR3 (SEQ ID NOS: 10, 11, 12, respectively); and/or
wherein the one or more anti-influenza antibodies binds to the same epitope as the antibody of (b1) and comprises a heavy and a light chain, wherein the heavy chain comprises: (b2) a polypeptide having at least 80% sequence identity with the TRL579 heavy chain CDR sequences HCDR1/HCDR2/HCDR3 (SEQ ID NOS: 7, 8, 9, respectively) and the TRL579 light chain CDR sequences LCDR1/LCDR2/LCDR3 (SEQ ID NOS: 10, 11, 12, respectively); and/or
wherein the one or more anti-influenza antibodies comprises: a heavy and a light chain comprising (c1) the heavy and the light chain CDR sequences, HCDR1/HCDR2/HCDR3, and LCDR1/LCDR2/LCDR3, respectively, selected from: (i) SEQ ID NOS: 13, 14, 15 and SEQ ID NOS: 16, 17, 18 (TRL809); (ii) SEQ ID NOS: 19, 20, 21 and SEQ ID NOS: 22, 23, 24 (TRL812); (iii) SEQ ID NOS: 25, 26, 27 and SEQ ID NOS: 28, 29, 30 (TRL813); (iv) SEQ ID NOS: 31, 32, 33 and SEQ ID NOS: 34, 35, 36 (TRL832); (v) SEQ ID NOS: 37, 38, 39 and SEQ ID NOS: 40, 41, 42 (TRL841); (vi) SEQ ID NOS: 43, 44, 45 and SEQ ID NOS: 46, 47, 48 (TRL842); (vii) SEQ ID NOS: 49, 50, 51 and SEQ ID NOS: 52, 53, 54 (TRL845); (viii) SEQ ID NOS: 55, 56, 57 and SEQ ID NOS: 58, 59, 60 (TRL846); (ix) SEQ ID NOS: 61, 62, 63 and SEQ ID NOS: 64, 65, 66 (TRL847); (x) SEQ ID NOS: 67, 68, 69 and SEQ ID NOS: 70, 71, 72 (TRL848); (xi) SEQ ID NOS: 73, 74, 75 and SEQ ID NOS: 76, 77, 78,(TRL849); (xii) SEQ ID NOS: 79; 80, 81 and SEQ ID NOS: 82, 83, 84 (TRL854); and (xiii) SEQ ID NOS: 85, 86, 87 and SEQ ID NOS: 88, 89, 90 (TRL856), respectively, and/or
wherein the one or more anti-influenza antibodies binds to the same epitope as the antibody of (c1) and comprises a heavy and a light chain, wherein the heavy chain comprises: (c2) a polypeptide having at least 80% sequence identity with the heavy and the light chain CDR sequences, HCDR1/HCDR2/HCDR3 and LCDR1/LCDR2/LCDR3, respectively, selected from: (i) SEQ ID NOS: 13, 14, 15 and SEQ ID NOS: 16, 17, 18 (TRL809); (ii) SEQ ID NOS: 19, 20, 21 and SEQ ID NOS: 22, 23, 24 (TRL812); (iii) SEQ ID NOS: 25, 26, 27 and SEQ ID NOS: 28, 29, 30 (TRL813); (iv) SEQ ID NOS: 31, 32, 33 and SEQ ID NOS: 34, 35, 36 (TRL832); (v) SEQ ID NOS: 37, 38, 39 and SEQ ID NOS: 40, 41, 42 (TRL841); (vi) SEQ ID NOS: 43, 44, 45 and SEQ ID NOS: 46, 47, 48 (TRL842); (vii) SEQ ID NOS: 49, 50, 51 and SEQ ID NOS: 52, 53, 54 (TRL845); (viii) SEQ ID NOS: 55, 56, 57 and SEQ ID NOS: 58, 59, 60 (TRL846); (ix) SEQ ID NOS: 61, 62, 63 and SEQ ID NOS: 64, 65, 66 (TRL847); (x) SEQ ID NOS: 67, 68, 69 and SEQ ID NOS: 70, 71, 72 (TRL848); (xi) SEQ ID NOS: 73, 74, 75 and SEQ ID NOS: 76, 77, 78 (TRL849); (xii) SEQ ID NOS: 79; 80, 81 and SEQ ID NOS: 82, 83, 84 (TRL854); and (xiii) SEQ ID NOS: 85, 86, 87 and SEQ ID NOS: 88, 89, 90 (TRL856), respectively.

13. The aqueous therapeutic protein formulation of claim 12, wherein the one or more anti-influenza antibodies comprises at least three anti-influenza antibodies each of which comprises a heavy and light chain, wherein
(i) the heavy and the light chain of the first antibody comprises: the TRL053 heavy chain CDR sequences HCDR1/HCDR2/HCDR3 (SEQ ID NOS: 1, 2, 3, respectively) and the TRL053 light chain CDR sequences LCDR1/LCDR2/LCDR3 (SEQ ID NOS: 4, 5, 6, respectively);
(ii) the heavy and the light chain of the second antibody comprises: the TRL579 heavy chain CDR sequences HCDR1/HCDR2/HCDR3 (SEQ ID NOS: 7, 8, 9, respectively) and the TRL579 light chain CDR sequences LCDR1/LCDR2/LCDR3 (SEQ ID NOS: 10, 11, 12, respectively); and
(iii) the heavy and the light chain of the third antibody comprise: the TRL849 heavy chain CDR sequences, HCDR1/HCDR2/HCDR3 (SEQ ID NOS: 73, 74, and 75, respectively) and the TRL849 light chain CDR sequences (LCDR1/LCDR2/LCDR3 of SEQ ID NOS: 76, 77, 78, respectively).

14. The aqueous therapeutic protein formulation of claim 1, wherein the one or more therapeutic proteins do not lose more than 15% of its biological activity during storage relative to an activity of the therapeutic protein at beginning of storage, wherein storage is at least for 3 month at 2° C-8° C.

15. The aqueous therapeutic protein formulation of claim 1, wherein the storage is for a period in the range of three month to two years.

16. The aqueous therapeutic formulation according to claim 1, wherein the aqueous therapeutic formulation is a pharmaceutical aqueous therapeutic formulation.

17. A method of generating an aerosol comprising the step of:
nebulizing the aqueous therapeutic protein formulation of claim 1, using a nebulizer to obtain an aerosol.

18. The method of claim 17, wherein the nebulizer is a vibrating membrane nebulizer.

19. A method for treating and preventing influenza, which method comprises administering a therapeutically effective amount of the pharmaceutical aqueous therapeutic protein formulation of claim 16 to a subject in need thereof, wherein the aqueous therapeutic protein formulation is administered intranasally or by inhalation.

20. The method of claim 19, wherein the administering comprises inhaling an aerosol generated by a nebulizer.

21. The method of claim 20, wherein the nebulizer is a vibrating mesh nebulizer.

22. The method of claim 20, wherein the nebulizer generates an aerosol targeting the lower respiratory tract of a subject.

23. The method of claim 20, wherein the nebulizer generates an aerosol targeting the upper respiratory tract of a subject.


SEQUENCE LISTING (123 sequences)

SEQ ID NO: 1 - protein, 8 aa, Artificial Sequence
  TRL053 HC CDR1
GGIIRKYA

SEQ ID NO: 2 - protein, 8 aa, Artificial Sequence
  TRL053 HC CDR2
IIAIFNTA

SEQ ID NO: 3 - protein, 13 aa, Artificial Sequence
  TRL053 HC CDR3
ARGMNYYSDYFDY

SEQ ID NO: 4 - protein, 7 aa, Artificial Sequence
  TRL053 LC CDR1
QSVRSNN

SEQ ID NO: 5 - protein, 3 aa, Artificial Sequence
  TRL053 LC CDR2
GAS

SEQ ID NO: 6 - protein, 10 aa, Artificial Sequence
  TRL053 LC CDR3
QQYGSSPALT

SEQ ID NO: 7 - protein, 8 aa, Artificial Sequence
  TRL579 HC CDR1
GYTFTAYT

SEQ ID NO: 8 - protein, 8 aa, Artificial Sequence
  TRL579 HC CDR2
INAGNGHT

SEQ ID NO: 9 - protein, 25 aa, Artificial Sequence
  TRL579 HC CDR3
ARGPETYYYDKTNWLNSHPDEYFQH

SEQ ID NO: 10 - protein, 6 aa, Artificial Sequence
  TRL579 LC CDR1
QTINNY

SEQ ID NO: 11 - protein, 3 aa, Artificial Sequence
  TRL579 LC CDR2
KAS

SEQ ID NO: 12 - protein, 10 aa, Artificial Sequence
  TRL579 LC CDR3
QEYNNDSPLT

SEQ ID NO: 13 - protein, 8 aa, Artificial Sequence
  TRL809 HC CDR1
GFTFSAYA

SEQ ID NO: 14 - protein, 8 aa, Artificial Sequence
  TRL809 HC CDR2
TSYDGRDS

SEQ ID NO: 15 - protein, 19 aa, Artificial Sequence
  TRL809 HC CDR3
AKVGPYTGYDDYNYYGMDV

SEQ ID NO: 16 - protein, 12 aa, Artificial Sequence
  TRL809 LC CDR1
QSVLSNSKNLNY

SEQ ID NO: 17 - protein, 3 aa, Artificial Sequence
  TRL809 LC CDR2
WAS

SEQ ID NO: 18 - protein, 9 aa, Artificial Sequence
  TRL809 LC CDR3
QQHYSIPLT

SEQ ID NO: 19 - protein, 8 aa, Artificial Sequence
  TRL812 HC CDR1
GFNFSSYG

SEQ ID NO: 20 - protein, 8 aa, Artificial Sequence
  TRL812 HC CDR2
IWSGGSNK

SEQ ID NO: 21 - protein, 15 aa, Artificial Sequence
  TRL812 HC CDR3
ARAHTALTRFRWLDP

SEQ ID NO: 22 - protein, 6 aa, Artificial Sequence
  TRL812 LC CDR1
QGIGSN

SEQ ID NO: 23 - protein, 3 aa, Artificial Sequence
  TRL812 LC CDR2
AAS

SEQ ID NO: 24 - protein, 9 aa, Artificial Sequence
  TRL812 LC CDR3
QQYNTWPLT

SEQ ID NO: 25 - protein, 8 aa, Artificial Sequence
  TRL813 HC CDR1
GFIFGDFA

SEQ ID NO: 26 - protein, 10 aa, Artificial Sequence
  TRL813 HC CDR2
IRSGLNRFET

SEQ ID NO: 27 - protein, 11 aa, Artificial Sequence
  TRL813 HC CDR3
TYLVGAVGFQH

SEQ ID NO: 28 - protein, 6 aa, Artificial Sequence
  TRL813 LC CDR1
QTIYRW

SEQ ID NO: 29 - protein, 3 aa, Artificial Sequence
  TRL813 LC CDR2
KAS

SEQ ID NO: 30 - protein, 9 aa, Artificial Sequence
  TRL813 LC CDR3
QQYNSFPYR

SEQ ID NO: 31 - protein, 8 aa, Artificial Sequence
  TRL832 HC CDR1
GGSVSNYY

SEQ ID NO: 32 - protein, 10 aa, Artificial Sequence
  TRL832 HC CDR2
VFYSGSSNYN

SEQ ID NO: 33 - protein, 19 aa, Artificial Sequence
  TRL832 HC CDR3
YCARGRVNSGYDWGPNFDS

SEQ ID NO: 34 - protein, 6 aa, Artificial Sequence
  TRL832 LC CDR1
RLGEKF

SEQ ID NO: 35 - protein, 3 aa, Artificial Sequence
  TRL832 LC CDR2
EDS

SEQ ID NO: 36 - protein, 9 aa, Artificial Sequence
  TRL832 LC CDR3
QAWDSSTVV

SEQ ID NO: 37 - protein, 8 aa, Artificial Sequence
  TRL841 HC CDR1
GFTFSSYG

SEQ ID NO: 38 - protein, 8 aa, Artificial Sequence
  TRL841 HC CDR2
ISYDEINK

SEQ ID NO: 39 - protein, 13 aa, Artificial Sequence
  TRL841 HC CDR3
AKPLRGSYRYFDL

SEQ ID NO: 40 - protein, 6 aa, Artificial Sequence
  TRL841 LC CDR1
QGLGSN

SEQ ID NO: 41 - protein, 3 aa, Artificial Sequence
  TRL841 LC CDR2
AAS

SEQ ID NO: 42 - protein, 9 aa, Artificial Sequence
  TRL841 LC CDR3
QQYTYWPLT

SEQ ID NO: 43 - protein, 8 aa, Artificial Sequence
  TRL842 HC CDR1
GFSLWTSG

SEQ ID NO: 44 - protein, 8 aa, Artificial Sequence
  TRL842 HC CDR2
MSYDETKK

SEQ ID NO: 45 - protein, 16 aa, Artificial Sequence
  TRL842 HC CDR3
AKPRLDYLDYFHAADS

SEQ ID NO: 46 - protein, 6 aa, Artificial Sequence
  TRL842 LC CDR1
YIGSKS

SEQ ID NO: 47 - protein, 3 aa, Artificial Sequence
  TRL842 LC CDR2
DDS

SEQ ID NO: 48 - protein, 12 aa, Artificial Sequence
  TRL842 LC CDR3
CQVWETSEDLWV

SEQ ID NO: 49 - protein, 8 aa, Artificial Sequence
  TRL845 HC CDR1
GFTFSRYS

SEQ ID NO: 50 - protein, 8 aa, Artificial Sequence
  TRL845 HC CDR2
ISDTSRTI

SEQ ID NO: 51 - protein, 14 aa, Artificial Sequence
  TRL845 HC CDR3
ARDPDTPFVRAFDS

SEQ ID NO: 52 - protein, 6 aa, Artificial Sequence
  TRL845 LC CDR1
QLISSY

SEQ ID NO: 53 - protein, 3 aa, Artificial Sequence
  TRL845 LC CDR2
AAS

SEQ ID NO: 54 - protein, 10 aa, Artificial Sequence
  TRL845 LC CDR3
QQLNSYPPIT

SEQ ID NO: 55 - protein, 8 aa, Artificial Sequence
  TRL846 HC CDR1
GFTFNSYA

SEQ ID NO: 56 - protein, 8 aa, Artificial Sequence
  TRL846 HC CDR2
IRGSGENT

SEQ ID NO: 57 - protein, 22 aa, Artificial Sequence
  TRL846 HC CDR3
ARKWGRMTVFGVATDQLYYFDY

SEQ ID NO: 58 - protein, 6 aa, Artificial Sequence
  TRL846 LC CDR1
QSVSSN

SEQ ID NO: 59 - protein, 3 aa, Artificial Sequence
  TRL846 LC CDR2
GAS

SEQ ID NO: 60 - protein, 10 aa, Artificial Sequence
  TRL846 LC CDR3
QQFNDWPFLT

SEQ ID NO: 61 - protein, 8 aa, Artificial Sequence
  TRL847 HC CDR1
GFTFSRFS

SEQ ID NO: 62 - protein, 8 aa, Artificial Sequence
  TRL847 HC CDR2
ISDTGRTI

SEQ ID NO: 63 - protein, 14 aa, Artificial Sequence
  TRL847 HC CDR3
ARDPDTPFVRAFDS

SEQ ID NO: 64 - protein, 6 aa, Artificial Sequence
  TRL847 LC CDR1
QVISSY

SEQ ID NO: 65 - protein, 3 aa, Artificial Sequence
  TRL847 LC CDR2
AAS

SEQ ID NO: 66 - protein, 10 aa, Artificial Sequence
  TRL847 LC CDR3
QQLTTYPPIT

SEQ ID NO: 67 - protein, 8 aa, Artificial Sequence
  TRL848 HC CDR1
GFSLWTSG

SEQ ID NO: 68 - protein, 8 aa, Artificial Sequence
  TRL848 HC CDR2
MSYDETKK

SEQ ID NO: 69 - protein, 16 aa, Artificial Sequence
  TRL848 HC CDR3
AKPRLDYLDYFHAADS

SEQ ID NO: 70 - protein, 6 aa, Artificial Sequence
  TRL848 LC CDR1
YIGSKS

SEQ ID NO: 71 - protein, 3 aa, Artificial Sequence
  TRL848 LC CDR2
DDS

SEQ ID NO: 72 - protein, 12 aa, Artificial Sequence
  TRL848 LC CDR3
CQVWETSEDLWV

SEQ ID NO: 73 - protein, 10 aa, Artificial Sequence
  TRL849 HC CDR1
GGSISNGGYH

SEQ ID NO: 74 - protein, 7 aa, Artificial Sequence
  TRL849 HC CDR2
IYYRGST

SEQ ID NO: 75 - protein, 21 aa, Artificial Sequence
  TRL849 HC CDR3
ARMPLANYDLLTGLYIGAFDL

SEQ ID NO: 76 - protein, 6 aa, Artificial Sequence
  TRL849 LC CDR1
QSVNRN

SEQ ID NO: 77 - protein, 3 aa, Artificial Sequence
  TRL849 LC CDR2
DAS

SEQ ID NO: 78 - protein, 9 aa, Artificial Sequence
  TRL849 LC CDR3
QQYDKWPPG

SEQ ID NO: 79 - protein, 8 aa, Artificial Sequence
  TRL854 HC CDR1
GYTFTAYH

SEQ ID NO: 80 - protein, 8 aa, Artificial Sequence
  TRL854 HC CDR2
INPNSGAT

SEQ ID NO: 81 - protein, 22 aa, Artificial Sequence
  TRL854 HC CDR3
ATDIVVERDASLGGFNSYGMDV

SEQ ID NO: 82 - protein, 11 aa, Artificial Sequence
  TRL854 LC CDR1
QSLLHSNGYNH

SEQ ID NO: 83 - protein, 3 aa, Artificial Sequence
  TRL854 LC CDR2
LAS

SEQ ID NO: 84 - protein, 9 aa, Artificial Sequence
  TRL854 LC CDR3
MQSLQTSIT

SEQ ID NO: 85 - protein, 8 aa, Artificial Sequence
  TRL856 HC CDR1
GFTFSDYW

SEQ ID NO: 86 - protein, 8 aa, Artificial Sequence
  TRL856 HC CDR2
ISSDGSTT

SEQ ID NO: 87 - protein, 15 aa, Artificial Sequence
  TRL856 HC CDR3
ARVATPYYYESGFDY

SEQ ID NO: 88 - protein, 6 aa, Artificial Sequence
  TRL856 LC CDR1
DIGSKS

SEQ ID NO: 89 - protein, 3 aa, Artificial Sequence
  TRL856 LC CDR2
DDR

SEQ ID NO: 90 - protein, 11 aa, Artificial Sequence
  TRL856 LC CDR3
QVWDITSDLVV

SEQ ID NO: 91 - protein, 105 aa, Homo sapiens [sequence omitted]

SEQ ID NO: 92 - protein, 104 aa, Homo sapiens
  VARIANT (1)..(104): Human LC Lambda constant Lambda constant
PKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVVPAECS

SEQ ID NO: 93 - protein, 330 aa, Homo sapiens [sequence omitted]

SEQ ID NO: 94 - protein, 450 aa, Artificial Sequence
  TRL053 HC
QVQLVQSGAEVRKPGSSVKVSCKVSGGIIRKYAINWVRQAPGQGLEWMGGIIAIFNTANYAQKFQGRVTITADESTSTVYMELSSLRSEDTALYYCARGMNYYSDYFDYWGQGSLVTVSPASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 95 - protein, 216 aa, Artificial Sequence
  TRL053 LC
EIVLTQSPGTLSLSPGERATLSCRASQSVRSNNLAWYQHKPGQAPRLLIFGASSRATGIPDRFSGSGSGTDFTLTISRLEPEDFAVYYCQQYGSSPALTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 96 - protein, 462 aa, Artificial Sequence
  TRL579 HC
QVQLVQSGAEVKKPGASVKVSCKTSGYTFTAYTIHWVRQAPGQRLEWMGWINAGNGHTKYSQRFKGRVTITRDTSARTTYMELRSLTSEDTALYFCARGPETYYYDKTNWLNSHPDEYFQHWGHGTQVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 97 - protein, 215 aa, Artificial Sequence
  TRL579 LC
DIQMTQSPSTLSASVGDRVTITCRASQTINNYLAWYQQKPGKAPKLLIYKASSLESGVPSRFSGSGSGTEFTLTISSLQPDDFATYYCQEYNNDSPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 98 - protein, 456 aa, Artificial Sequence
  TRL809 HC
QVQLLESGGGVVRPGRSLRLACAGSGFTFSAYAMHWVRQAPGKGLEWVAHTSYDGRDSYYVDSVKGRFTISRDDSKNTLYLQMNSLRPEDTAVYYCAKVGPYTGYDDYNYYGMDVWGHGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 99 - protein, 220 aa, Artificial Sequence
  TRL809 LC
DIVLTQSPVSLAVSLGESATVNCKSSQSVLSNSKNLNYLAWYQHKPGQPPKLLIYWASTRESGVPDRFSGSGSGTDFTLTISSLQAEDVAVYYCQQHYSIPLTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 100 - protein, 452 aa, Artificial Sequence
  TRL812 HC
QVQLVQSGGGVVQPGRSLRLSCAASGFNFSSYGMHWVRQAPGKGLEWLSVIWSGGSNKYYADSVKGRFTISRDDSKNTVYLQMNSLRDEDTAVYYCARAHTALTRFRWLDPWGQGILVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 101 - protein, 214 aa, Artificial Sequence
  TRL812 LC
DIVMTQSPATLSVSPGEGATLSCRASQGIGSNLAWYQHKPGQAPRLLIYAASTRATGVPARFSGSGSGTEFTLTISSLQSEDLAVYFCQQYNTWPLTFGGGTKVEIIRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 102 - protein, 450 aa, Artificial Sequence
  TRL813 HC
QVQLVESGGGLVQPGGSLRLSCAASGFIFGDFAIHWVRQASGKGLEWVARIRSGLNRFETAYAASVEGRLTISRDDSKSMAYLQMNNVKAEDTAVYLCTYLVGAVGFQHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 103 - protein, 214 aa, Artificial Sequence
  TRL813 LC
DIVMTQSPSTLSASVGDRVTITCRASQTIYRWLAWYQQKPGKAPKVLIYKASTLESGVPSRFSGSGSGTEFTLAISSLQPDDFATYYCQQYNSFPYRFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 104 - protein, 458 aa, Artificial Sequence
  TRL832 HC
QVQLVESGPGLMKPSETLSLTCTVSGGSVSNYYWSWIRQPPGKGLEWIGYVFYSGSSNYNPSLNNPSLKSRVTISIDTSNNQFSLKLSSVTAADTAVYYCARGRVNSGYDWGPNFDSWGQGALVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 105 - protein, 212 aa, Artificial Sequence
  TRL832 LC
DIVLTQAPSVSVSPGQTASITCSGDRLGEKFASWYQQKPGQSPVLVIYEDSKRPSGIPDRFSGSNSGNTATLTISGTQIMDEGDFFCQAWDSSTVVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVVPAECS

SEQ ID NO: 106 - protein, 450 aa, Artificial Sequence
  TRL841 HC
QVQLVESGGGVVQPGRSLRLSCAASGFTFSSYGIYWVRQAPGKGLEWVAFISYDEINKFYADSVKGRFTISRDNSKNTVFLQMNSLRDEDTAVYYCAKPLRGSYRYFDLWGRGTVVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 107 - protein, 214 aa, Artificial Sequence
  TRL841 LC
DIVMTQPPATLSVSPGERATLSCGASQGLGSNLAWYQQKPGQAPRLLIYAASTRATGITARFSGSGSGTEFTLTINSLQSEDFALYYCQQYTYWPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 108 - protein, 453 aa, Artificial Sequence
  TRL842 HC
QVQLLESGGDVVQPGTSLRLSCAASGFSLWTSGMHWVRQAPGKGLEWVAVMSYDETKKYYADSVRGRFSISRDNSRNTLYLQMNSLRVEDTAVYFCAKPRLDYLDYFHAADSWGQGTLVSVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 109 - protein, 217 aa, Artificial Sequence
  TRL842 LC
DIVLTQPPSVSVAPGQTATITCGGNYIGSKSVNWYQQRPGHAPVLVVYDDSDRPSGIPDRFSGSNSRSTATTATLTISRVEAGDEADYYCQVWETSEDLWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVVPAECS

SEQ ID NO: 110 - protein, 451 aa, Artificial Sequence
  TRL845 HC
QVQLVQSGGDLVQPGGSLRLSCAASGFTFSRYSMNWVRQAPGMGLEWVAYISDTSRTIYYADSVKGRFTISRDNAKNSLYLQMKSLRDEDTAVYYCARDPDTPFVRAFDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 111 - protein, 215 aa, Artificial Sequence
  TRL845 LC
DIVLTQSPSFLSASVGDRVTITCRASQLISSYFAWYQQKPGKPPKLLIYAASTLQSGVPSRFSGSGSGTQFSLTISSLQPEDFATYYCQQLNSYPPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 112 - protein, 459 aa, Artificial Sequence
  TRL846 HC
QVQLVESGGGLVQPGGSLRLSCTVSGFTFNSYAMNWVRQAPGKGLEWVSGIRGSGENTYYAETVKGRFTISRDNSKNTLYLEMNSLRAEDTAVYYCARKWGRMTVFGVATDQLYYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 113 - protein, 215 aa, Artificial Sequence
  TRL846 LC
DITLTQSPATLSVSPGERATLSCRASQSVSSNLAWYQQKPGQAPRLLIYGASNRATDIPARFSGSGSGTEFTLTISSLQSEDFAVYYCQQFNDWPFLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 114 - protein, 451 aa, Artificial Sequence
  TRL847 HC
QVQLVESGGGLVQPGGSLRLSCVASGFTFSRFSMNWVRQAPGMGLEWVSYISDTGRTIYYADSVKGRFTISRDNDKNTLYLQMKSLRDEDSAFYYCARDPDTPFVRAFDSWGQGVLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 115 - protein, 215 aa, Artificial Sequence
  TRL847 LC
DIVMTQSPSFLSASVGDRVTITCRTSQVISSYLAWYQQRPGKAPKLLIYAASTLQSGVPSRFSGSGSGTRFSLTISNLQPEDFATYFCQQLTTYPPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 116 - protein, 453 aa, Artificial Sequence; identical to SEQ ID NO: 108
  TRL848 HC

SEQ ID NO: 117 - protein, 217 aa, Artificial Sequence; identical to SEQ ID NO: 109
  TRL848 LC

SEQ ID NO: 118 - protein, 459 aa, Artificial Sequence
  TRL849 HC
QVQLVESGPGLVKPSQTLSLTCTVSGGSISNGGYHWSWIRQHPGKGLEWIGNIYYRGSTFYDPSLKSRVTISVDTSKSQFSLKLTSVTAADTAVYYCARMPLANYDLLTGLYIGAFDLWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 119 - protein, 214 aa, Artificial Sequence
  TRL849 LC
DIVLTQSPATLSVSPGERATLSCRASQSVNRNLAWFQQIPGQAPRLLIFDASTRAADIPARFSGGGSGAEFTLTVSSLQPEDFAVYYCQQYDKWPPGFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 120 - protein, 459 aa, Artificial Sequence
  TRL854 HC
QVQLVESGAEVKKPGASVRVSCKGSGYTFTAYHMHWVRQAPGQGFEWVGWINPNSGATYYTSTFEGRVTMTRDTSITTAYMELSSLRSDDTAVYYCATDIVVERDASLGGFNSYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 121 - protein, 219 aa, Artificial Sequence
  TRL854 LC
DIEMTQSPLSLPVTPGEPASISCRSSQSLLHSNGYNHLAWYLQKPGRSPHLLIYLASTRASGVPDRFSGSGSGTDFTLKISRVEAEDVGIYYCMQSLQTSITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 122 - protein, 452 aa, Artificial Sequence
  TRL856 HC
QVQLVESGGGLVQPGGSLRLSCAASGFTFSDYWMVWVRQAPGKGLVWVSRISSDGSTTSYADSVKGPFTISRDNAKNTLYLQMNSLRAEDTAVYYCARVATPYYYESGFDYWGQGALVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 123 - protein, 214 aa, Artificial Sequence
  TRL856 LC
DIVLTQSPSVSVAPGQTARITCGGNDIGSKSVHWYQQKPGQAPVLVVFDDRDRPSGIPERFSGSNSGNTATLTISRVEAGDEADYYCQVWDITSDLVVFGGGTKLTVVGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVVPAECS